(19) United States
(12) Patent Application Publication
(10) Pub. No.: US 2022/0056115 A1
(43) Pub. Date: Feb. 24, 2022

(54) ADMINISTRATION OF AN ANTI-C5 AGENT FOR TREATMENT OF HEPATIC INJURY OR FAILURE

(71) Applicant: Kyoto University, Kyoto (JP)

(72) Inventors: Koichiro HATA, Kyoto (JP); Jiro KUSAKABE, Kyoto (JP)

(21) Appl. No.: 17/274,858

(22) PCT Filed: Sep. 16, 2019
(86) PCT No.: PCT/IB2019/001023

§ 371 Date: Mar. 10, 2021

Related U.S. Application Data
(60) Provisional application No. 62/732,459, filed on Sep. 17, 2018.

(51) Int. Cl. C07K 16/18; A61P 1/16

(52) U.S. Cl. CPC C07K 16/18; A61K 2039/505; C07K 2317/565; A61P 1/16

(57) ABSTRACT

Provided herein are methods for treating liver injury (e.g., hepatic ischemia reperfusion injury (IRI)) or liver failure (e.g., acute liver failure) in a patient, comprising administering to the patient an anti-C5 agent (e.g., anti-C5 antibody, or antigen binding fragment thereof, such as eculizumab or ravulizumab). Also provided are methods for decreasing levels of one or more pro-inflammatory cytokines and/or one or more chemokines, decreasing neutrophil infiltration, increasing serum albumin, decreasing Prothrombin Time (PT), and decreasing International Normalized Ratio (INR) in a patient by administering to the patient an anti-C5 agent, such as an antibody, or antigen binding fragment thereof.

### CROSS-REFERENCE TO RELATED APPLICATIONS

This application claims the benefit of U.S. Provisional Application Ser. No. 62/732,459, filed on Sep. 17, 2018. The entire contents of the provisional patent application is incorporated herein by reference.

### SEQUENCE LISTING

The instant application contains a Sequence Listing which has been submitted electronically in ASCII format and is hereby incorporated by reference in its entirety. Said ASCII copy, created on Sep. 12, 2019, is named AXJ-255PC_SL.txt and is 58,780 bytes in size.

### BACKGROUND

Ischemia reperfusion injury (IRI) is a phenomenon during which cellular damage in an organ, caused by hypoxia (oxygen deficiency in a tissue), is exacerbated after the restoration of oxygen delivery (see Papadopoulos, et al., Arch Trauma Res. 2013 August; 2(2): 63-70 and Elias-Miro M, et al., Ischemia-Reperfusion Injury Associated with Liver Transplantation in 2011: Past and Future. 2012). If severe enough, the inflammatory response after IRI can result in systemic inflammatory response syndrome (SIRS) or multiple organ dysfunction syndrome (MODS) (see Videla L A, et al., World J Hepatol. 2009; 1(1):72-8). Hepatic IRI occurs in the setting of transplantation, trauma, shock, and elective liver surgery, in which hepatic blood supply is temporarily interrupted.

Acute liver failure (also known as fulminant hepatic failure) is a loss of liver function (e.g., loss of function of 80-90% of liver cells) that occurs rapidly (e.g., in days or weeks), usually in a person who has no pre-existing liver disease. Acute liver failure is defined as a syndrome of acute hepatitis with evidence of abnormal coagulation (e.g., an international normalized ratio >1.5) complicated by the development of mental alteration (encephalopathy) within 26 weeks of the onset of illness in a patient without a history of liver disease (see, e.g., Polson J, Lee W M; American Association for the Study of Liver Disease. Hepatology 2005; 41:1179-1197 and Sleisenger & Fordtran's gastrointestinal and liver disease pathophysiology, diagnosis, management (PDF) (9th ed.)).

There are nearly 2,000 cases of acute liver failure each year in the United States, and it accounts for 6% of all deaths due to liver disease (see Singh et al., Cleveland Clinic Journal of Medicine. 2016 June; 83(6):453-462 and Lee W M, et al., Acute liver failure: summary of a workshop. Hepatology 2008; 47:1401-1415). This disease carries a high mortality rate, and early recognition and transfer to a tertiary medical care center with transplant facilities is critical. Hepatic IRI is a frequent and major complication in clinical practice, which compromises liver function and increases postoperative morbidity, mortality, recovery, and overall outcome. Accordingly, it is an object of the present invention to provide improved methods for treating patients with hepatic IRI (e.g., due to any type of physical injury, including surgery), as well as patients determined to have acute liver failure.

### SUMMARY

Provided herein are compositions and methods for treating liver injury or failure in a patient, comprising administering to the patient an anti-C5 agent, such as a polypeptide, a polypeptide analog, a nucleic acid, a nucleic acid analog, and a small molecule. An exemplary anti-C5 agent is an anti-C5 antibody, or antigen binding fragment thereof.

In one aspect, a method of treating hepatic ischemia reperfusion injury (IRI) in a patient who has experienced hepatic trauma (e.g., due to any type of physical injury, including surgery), comprising administering to the patient an effective amount of an anti-C5 agent, such as an anti-C5 antibody, or antigen binding fragment thereof. In one embodiment, the treatment results in decreased hepatocyte apoptosis compared to a pre-treatment baseline (e.g., as assessed by single-stranded-DNA staining and/or western blot for cleaved caspase-3). In one embodiment, the treatment results in a 1.5-fold, 2-fold, 2.5-fold, 3-fold, or 3.5 fold decrease in hepatocyte apoptosis compared to a pre-treatment baseline.

In another embodiment, the treatment results in a decrease in one or more pro-inflammatory cytokines (e.g., IL-1β, IL-6, and/or TNFα) and/or one or more chemokines (e.g., CXCL-1 and/or CXCL-2) compared to a pre-treatment baseline (e.g., a 25%, 30% 40%, 50%, 60%, 70%, 80% or greater decrease). In another embodiment, the treatment results in a decrease in one or more pro-inflammatory cytokines (e.g., IL-1β, IL-6, and/or TNFα) and/or one or more chemokines (e.g., CXCL-1 and/or CXCL-2) to within normal levels or to within 10%, 15%, or 20% above what is considered the normal level.

In another embodiment, the treatment results in a decrease in one or more parenchymal damage markers (e.g., AST, ALT and/or T-bil) compared to a pre-treatment baseline (e.g., a 25%, 30% 40%, 50%, 60%, 70%, 80% or greater decrease). In another embodiment, the treatment results in a decrease in one or more parenchymal damage markers (e.g., AST, ALT and/or T-bil) to within normal levels or to within 10%, 15%, or 20% above what is considered a normal level for the marker.

In another embodiment, the treatment results in decreased neutrophil infiltration compared to a pre-treatment baseline (e.g., a 25%, 30% 40%, 50%, 60%, 70%, 80% or greater decrease). In another embodiment, the treatment results in decreased neutrophil infiltration to within normal levels of neutrophils or to within 10%, 15%, or 20% above what is considered the normal level.

In another embodiment, the treatment results in decreased platelet aggregation compared to a pre-treatment baseline (e.g., a 25%, 30% 40%, 50%, 60%, 70%, 80% or greater decrease). In another embodiment, the treatment results in decreased platelet aggregation to within normal levels of platelet aggregation or to within 10%, 15%, or 20% above what is considered the normal level.

In another embodiment, the treatment results in an at least one score improvement, as assessed by the Suzuki Scoring System for the assessment of liver damage following hepatic IRI set forth in Table 1 (see, e.g., Matthias Behrends, et al., J Gastrointest Surg. 2010 March; 14(3): 528-535, Suzuki S, et al., Transplantation, 1993; 55(6): 1265-72, and Suzuki S, et al., Transplantation. 1991; 52:979-98). For example, the patient may have (1) a score of 4 prior to treatment and a score of 3 after treatment, (2) a score of 3 prior to treatment and a score of 2 after treatment, (3) a score of 2 prior to treatment and a score of 1 after treatment, or (4) a score of 1 prior to treatment and a score of 0 after treatment. In another embodiment, the treatment results in at least a 2,3, or 4 score improvement. For example, the patient may have (1) a score of 4 prior to treatment and a score of 2 after treatment, (2) a score of 3 prior to treatment and a score of 1 after treatment, (3) a score of 2 prior to treatment and a score of 0 after treatment, (4) a score of 4 prior to treatment and a score of 1 after treatment, (5) a score of 3 prior to treatment and a score of 0 after treatment, or (6) a score of 4 prior to treatment and a score of 0 after treatment.

Also provided are methods for decreasing levels of one or more pro-inflammatory cytokines (e.g., IL-1β, IL-6, and/or TNFα) and/or one or more chemokines (e.g., CXCL-1 and/or CXCL-2) in a patient who has experienced hepatic trauma, by administering to the patient an effective amount of an anti-C5 agent, such as an anti-C5 antibody, or antigen binding fragment thereof, thereby decreasing levels of the one or more pro-inflammatory cytokines and/or one or more chemokines in the patient compared to pre-treatment baseline levels. In one embodiment, the method results in a decrease in one or more pro-inflammatory cytokines (e.g., IL-1β, IL-6, and/or TNFα) and/or one or more chemokines (e.g., CXCL-1 and/or CXCL-2) compared to a pre-treatment baseline (e.g., a 25%, 30% 40%, 50%, 60%, 70%, 80% or greater decrease). In another embodiment, the method results in a decrease in one or more pro-inflammatory cytokines (e.g., IL-1β, IL-6, and/or TNFα) and/or one or more chemokines (e.g., CXCL-1 and/or CXCL-2) to within normal levels or to within 10%, 15%, or 20% above what is considered the normal level.

Further provided are methods of decreasing neutrophil infiltration in a patient who has experienced hepatic trauma (e.g., due to any type of physical injury, including surgery), by administering to the patient an effective amount of an anti-C5 agent, such as an anti-C5 antibody, or antigen binding fragment thereof, thereby decreasing neutrophil levels compared to pre-treatment baseline neutrophil levels. In one embodiment, the method results in decreased neutrophil infiltration compared to a pre-treatment baseline (e.g., a 25%, 30% 40%, 50%, 60%, 70%, 80% or greater decrease). In another embodiment, the treatment results in decreased neutrophil infiltration to within normal levels of neutrophils or to within 10%, 15%, or 20% above what is considered the normal level.

In another aspect, methods of treating a patient who has been determined to have acute liver failure are provided, comprising administering to the patient an effective amount of an anti-C5 agent, such as an anti-C5 antibody, or antigen binding fragment thereof. In one embodiment, the treatment results in a shift towards normal levels of serum albumin. In another embodiment, the treatment results in the patient having a Prothrombin Time (PT) between 9.5 to 13.5 seconds. In another embodiment, the treatment results in the patient having an International Normalized Ratio (INR) between 0.8 to 1.1. In another embodiment, the treatment results in the patient having a PT between 9.5 to 13.5 seconds and an INR between 0.8 to 1.1.

In one embodiment, the treatment produces at least one therapeutic effect selected from the group consisting of a reduction or cessation in hepatic encephalopathy, impaired protein synthesis, jaundice, pain in the upper right abdomen, abdominal swelling, nausea, vomiting, malaise, disorientation, confusion, and/or sleepiness. In another embodiment, the treatment produces a change from baseline, as assessed via The King's College criteria system, the Model for End-Stage Liver Disease (MELD) scoring system, the Acute Physiology and Chronic Health Evaluation (APACHE) II scoring system, and/or the Clichy criteria.

In a further aspect, methods of increasing serum albumin in a patient who has been determined to have acute liver failure are provided, comprising administering to the patient an effective amount of an anti-C5 agent, such as an anti-C5 antibody, or antigen binding fragment thereof, thereby increasing serum albumin in the patient compared to a pre-administration baseline serum albumin level. In one embodiment the patient's serum albumin is below 3.4 grams per deciliter prior to administration of the anti-C5 agent (e.g., anti-C5 antibody, or antigen binding fragment thereof). In another embodiment, the patient's serum albumin is between 3.4 grams to 5.4 grams per deciliter after administration of the anti-C5 agent (e.g., anti-C5 antibody, or antigen binding fragment thereof).

In a further aspect, methods of decreasing PT in a patient who has been determined to have acute liver failure are provided, comprising administering to the patient an effective amount of an anti-C5 agent (e.g., anti-C5 antibody, or antigen binding fragment thereof), thereby decreasing PT time in the patient compared. In one embodiment, the patient's PT is >13.5 seconds prior to administration of the anti-C5 agent (e.g., anti-C5 antibody, or antigen binding fragment thereof). In another embodiment, the patient's PT is between is 9.5 to 13.5 seconds after administration of the anti-C5 agent (e.g., anti-C5 antibody, or antigen binding fragment thereof).

In a further aspect, methods of decreasing INR in a patient who has been determined to have acute liver failure, the method comprising administering to the patient an effective amount of an anti-C5 agent (e.g., anti-C5 antibody, or antigen binding fragment thereof), thereby decreasing INR in the patient. In one embodiment, the patient's INR is >1.5 prior to administration of the anti-C5 agent (e.g., anti-C5 antibody, or antigen binding fragment thereof). In another embodiment, the patient's INR is between 0.8 to 1.1 after administration of the anti-C5 agent (e.g., anti-C5 antibody, or antigen binding fragment thereof).

The assessments described herein, for example, a decrease in hepatocyte apoptosis, cytokines, chemokines, parenchymal damage markers, neutrophil infiltration, platelet aggregation, PT, INR, and/or physical symptoms) can be determined at any time after administration of the anti-C5 agent (e.g., anti-C5 antibody, or antigen binding fragment thereof). In one embodiment, the decrease is assessed 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 48, or 72 hours post treatment.

Any suitable anti-C5 agent can be used in the methods described herein. In one embodiment, the anti-C5 agent is a polypeptide, a polypeptide analog, a nucleic acid, a nucleic acid analog, or a small molecule. In another embodiment, the agent-C5 agent is an anti-C5 antibody, or antigen binding fragment thereof. An exemplary anti-C5 antibody is eculizumab (Soliris®) comprising the heavy and light chains having the sequences shown in SEQ ID NOs:10 and 11, respectively, or antigen binding fragments and variants thereof. In other embodiments, the antibody comprises the heavy and light chain complementarity determining regions (CDRs) or variable regions (VRs) of eculizumab. Accordingly, in one embodiment, the antibody comprises the CDR1, CDR2, and CDR3 domains of the heavy chain variable (VH) region of eculizumab having the sequence shown in SEQ ID NO:7, and the CDR1, CDR2 and CDR3 domains of the light chain variable (VL) region of eculizumab having the sequence shown in SEQ ID NO:8. In another embodiment, the antibody comprises CDR1, CDR2 and CDR3 heavy chain sequences as set forth in SEQ ID NOs:1, 2, and 3, respectively, and CDR1, CDR2 and CDR3 light chain sequences as set forth in SEQ ID NOs:4, 5, and 6, respectively. In another embodiment, the antibody comprises VH and VL regions having the amino acid sequences set forth in SEQ ID NO:7 and SEQ ID NO: 8, respectively. In another embodiment, the antibody comprises a heavy chain as set forth in SEQ ID NO:10 and a light chain polypeptide as set forth in SEQ ID NO:11.

Another exemplary antibody is ravulizumab (also known as ALXN1210 and antibody BNJ441) comprising the heavy and light chains having the sequences shown in SEQ ID NOs:14 and 11, respectively, or antigen binding fragments and variants thereof. In other embodiments, the antibody comprises the heavy and light chain complementarity determining regions (CDRs) or variable regions (VRs) of ravulizumab. Accordingly, in one embodiment, the antibody comprises the CDR1, CDR2, and CDR3 domains of the heavy chain variable (VH) region of ravulizumab having the sequence shown in SEQ ID NO:12, and the CDR1, CDR2 and CDR3 domains of the light chain variable (VL) region of ravulizumab having the sequence shown in SEQ ID NO:8. In another embodiment, the antibody comprises CDR1, CDR2 and CDR3 heavy chain sequences as set forth in SEQ ID NOs:19, 18, and 3, respectively, and CDR1, CDR2 and CDR3 light chain sequences as set forth in SEQ ID NOs:4, 5, and 6, respectively.

In another embodiment, the antibody comprises VH and VL regions having the amino acid sequences set forth in SEQ ID NO: 12 and SEQ ID NO: 8, respectively. In another embodiment, the antibody comprises a heavy chain constant region as set forth in SEQ ID NO:13. In another embodiment, the antibody comprises a heavy chain polypeptide as set forth in SEQ ID NO:14 and a light chain polypeptide as set forth in SEQ ID NO:11. In another embodiment, the antibody comprises a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434 of a native human IgG Fc constant region, each in EU numbering.

In another embodiment, the antibody comprises CDR1, CDR2 and CDR3 heavy chain sequences as set forth in SEQ ID NOs:19, 18, and 3, respectively, and CDR1, CDR2 and CDR3 light chain sequences as set forth in SEQ ID NOs:4, 5, and 6, respectively and a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434 of a native human IgG Fc constant region, each in EU numbering.

In another embodiment, the antibody binds to human C5 at pH 7.4 and 25° C. with an affinity dissociation constant (K_{D}) that is in the range 0.1 nM≤K_{D}≤1 nM. In another embodiment, the antibody binds to human C5 at pH 6.0 and 25° C. with a K_{D}≥10 nM. In yet another embodiment, the [(K_{D} of the antibody or antigen-binding fragment thereof for human C5 at pH 6.0 and at 25° C.)/(K_{D} of the antibody or antigen-binding fragment thereof for human C5 at pH 7.4 and at 25° C.)] of the antibody is greater than 25.

Another exemplary anti-C5 antibody is the 7086 antibody described in U.S. Pat. Nos. 8,241,628 and 8,883,158. In one embodiment, the antibody comprises the heavy and light chain CDRs or variable regions of the 7086 antibody (see U.S. Pat. Nos. 8,241,628 and 8,883,158). In another embodiment, the antibody, or antigen binding fragment thereof, comprises heavy chain CDR1, CDR2 and CDR3 domains having the sequences set forth in SEQ ID NOs: 21, 22, and 23, respectively, and light chain CDR1, CDR2 and CDR3 domains having the sequences set forth in SEQ ID NOs: 24, 25, and 26, respectively. In another embodiment, the antibody, or antigen binding fragment thereof, comprises the VH region of the 7086 antibody having the sequence set forth in SEQ ID NO:27, and the VL region of the 7086 antibody having the sequence set forth in SEQ ID NO:28.

Another exemplary anti-C5 antibody is the 8110 antibody also described in U.S. Pat. Nos. 8,241,628 and 8,883,158. In one embodiment, the antibody comprises the heavy and light chain CDRs or variable regions of the 8110 antibody. In another embodiment, the antibody, or antigen binding fragment thereof, comprises heavy chain CDR1, CDR2 and CDR3 domains having the sequences set forth in SEQ ID NOs: 29, 30, and 31, respectively, and light chain CDR1, CDR2 and CDR3 domains having the sequences set forth in SEQ ID NOs: 32, 33, and 34, respectively. In another embodiment, the antibody comprises the VH region of the 8110 antibody having the sequence set forth in SEQ ID NO: 35, and the VL region of the 8110 antibody having the sequence set forth in SEQ ID NO: 36.

Another exemplary anti-C5 antibody is the 305L05 antibody described in US2016/0176954A1. In one embodiment, the antibody comprises the heavy and light chain CDRs or variable regions of the 305L05 antibody. In another embodiment, the antibody, or antigen binding fragment thereof, comprises heavy chain CDR1, CDR2 and CDR3 domains having the sequences set forth in SEQ ID NOs: 37, 38, and 39, respectively, and light chain CDR1, CDR2 and CDR3 domains having the sequences set forth in SEQ ID NOs: 40, 41, and 42, respectively. In another embodiment, the antibody comprises the VH region of the 305L05 antibody having the sequence set forth in SEQ ID NO: 43, and the VL region of the 305L05 antibody having the sequence set forth in SEQ ID NO: 44.

Another exemplary anti-C5 antibody is the SKY59 antibody described in Fukuzawa T., et al., Rep. 2017 Apr. 24; 7(1):1080). In one embodiment, the antibody comprises the heavy and light chain CDRs or variable regions of the SKY59 antibody. In another embodiment, the antibody, or antigen binding fragment thereof, comprises a heavy chain comprising SEQ ID NO: 45 and a light chain comprising SEQ ID NO: 46.

Another exemplary anti-C5 antibody is the REGN3918 antibody (also known as H4H12166PP) described in US20170355757. In one embodiment, the antibody comprises a heavy chain variable region comprising SEQ ID NO:47 and a light chain variable region comprising SEQ ID NO:48. In another embodiment, the antibody comprises a heavy chain comprising SEQ ID NO:49 and a light chain comprising SEQ ID NO:50.

In another embodiment, the antibody competes for binding with, and/or binds to the same epitope on C5 as, the above-mentioned antibodies (e.g., eculizumab, ravulizumab, 7086 antibody, 8110 antibody, 305L05 antibody, SKY59 antibody, or REGN3918 antibody). In another embodiment, the antibody has at least about 90% variable region amino acid sequence identity with the above-mentioned antibodies (e.g., at least about 90%, 91%, 92%, 93%, 94%, 95%, 96%, 97%, 98% or 99% variable region identity).

The anti-C5 agent (e.g., antibody, or antigen binding fragment thereof), can be administered to a patient by any suitable means. In one embodiment, the agent is administered intravenously. In another embodiment, the agent is administered orally. In another embodiment, the agent is administered, subcutaneously. In another embodiment, the agent is an anti-C5 antibody, or antigen binding fragment thereof, administered at a dose of about 400 mg, 405 mg, 410 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, 800 mg, 805 mg, 810 mg, 815 mg, 820 mg, 825 mg, 830 mg, 835 mg, 840 mg, 845 mg, 850 mg, 855 mg, 860 mg, 865 mg, 870 mg, 875 mg, 880 mg, 885 mg, 890 mg, 895 mg, 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, 1000 mg, 1005 mg, 1010 mg, 1015 mg, 1020 mg, 1025 mg, 1030 mg, 1035 mg, 1040 mg, 1045 mg, 1050 mg, 1055 mg, 1060 mg, 1065 mg, 1070 mg, 1075 mg, 1080 mg, 1085 mg, 1090 mg, 1095 mg, 1100 mg, 1150 mg, 1200 mg, 1250 mg, 1300 mg, 1350 mg, or 1400 mg. In one embodiment, the anti-C5 agent (e.g., antibody, or antigen binding fragment thereof), is administered in a single dose. In another embodiment, multiple doses of the anti-C5 agent (e.g., anti-C5 antibody, or antigen binding fragment thereof), are administered.

The anti-C5 agent (e.g., anti-C5 antibody, or antigen binding fragment thereof), can be administered alone or in combination (e.g., separately or simultaneously) with one or more additional therapeutic agents. In one embodiment, the anti-C5 agent (e.g., anti-C5 antibody, or antigen binding fragment thereof, is administered in combination with no more than three additional agents. In another embodiment, the anti-C5 agent (e.g., anti-C5 antibody, or antigen binding fragment thereof), is administered in combination with no more than two additional agents. In another embodiment, the anti-C5 agent (e.g., anti-C5 antibody, or antigen binding fragment thereof), is administered in combination with no more than one additional agent. In another embodiment, the anti-C5 agent (e.g., anti-C5 antibody, or antigen binding fragment thereof), is administered alone.

Also provided are kits that include a pharmaceutical composition containing an anti-C5 agent (e.g., an anti-C5 antibody, or antigen binding fragment thereof, such as eculizumab or ravulizumab), and a pharmaceutically-acceptable carrier, in a therapeutically effective amount adapted for use in the methods described herein. For example, in one embodiment, a kit for treating or preventing hepatic ischemia reperfusion injury (IRI) in a patient is provided, the kit comprising: (a) a dose of an anti-C5 agent (e.g., anti-C5 antibody, or antigen binding fragment thereof); and (b) instructions for using the anti-C5 agent (e.g., anti-C5 antibody, or antigen binding fragment thereof), in any of the methods described herein. In another embodiment, a kit for treating a patient who has been determined to have acute liver failure is provided, the kit comprising: (a) a dose of an anti-C5 agent (e.g., anti-C5 antibody, or antigen binding fragment thereof); and (b) instructions for using the anti-C5 agent (e.g., anti-C5 antibody, or antigen binding fragment thereof), in any of the methods described herein.

### BRIEF DESCRIPTION OF THE DRAWINGS

FIG. 1 is a schematic depicting the murine model of warm ischemia-reperfusion injury.

FIGS. 2A and 2B depict the effect of BB5.1 on CH50 administered intraperitoneally (i.p.) (FIG. 2A) or intravenously (i.v.) (FIG. 2B) to C5 WT mice.

FIG. 3 depicts hemolytic activity (CH50 U/ml) during ischemia, during reperfusion, 2 hours post IRI, 6 hours post IRI, 1 day post IRI, 3 days post IRI, and 4 days post IRI, with and without administration of an anti-C5 antibody.

FIG. 4 depicts ALT (U/ml) release after hepatic ischemia-reperfusion for up to 24 hours for WT-control IgG, WT-Anti-C5 Ab, KO-Control IgG, and KO-Anti-C5 Ab.

FIGS. 5A and 5B depict ALT release (U/ml) at 2 hours (FIG. 5A) and 6 hours (FIG. 5B) after hepatic ischemia-reperfusion for WT-control IgG, WT-Anti-C5 Ab, KO-Control IgG, KO-Anti-C5 Ab, WT-C5aR-Ant, WT-Sham, and KO-Sham.

FIGS. 6A and 6B depict the histopathological evaluation as assessed by Suzuki Score for WT-control IgG, WT-Anti-C5 Ab, KO-Control IgG, KO-Anti-C5 Ab, and WT-C5aR-Ant at 2 hours (FIG. 6A) and 6 hours (FIG. 6B) post ischemia-reperfusion.

FIG. 7 depicts CD41 staining for WT-control IgG, WT-Anti-C5 Ab, KO-Control IgG, KO-Anti-C5 Ab, and WT-C5aR-Ant2 hours post IRI.

FIGS. 8A-8J depict IL-10 (FIG. 8A and FIG. 8F), IL-6 (FIG. 8B and FIG. 8G), TNF-α (FIG. 8C and FIG. 8H), CXCL-1 (FIG. 8D and FIG. 8I), and CXCL-2 (FIG. 8E and FIG. 8J, as assessed by qRT-PCR, at 2 hour and 6 hours post ischemia-reperfusion.

FIGS. 9A-9D depict F4/80+ cells (whole liver macrophage) (FIG. 9A and FIG. 9B) and CD11b cells (infiltrating macrophages) (FIG. 9C and FIG. 9D) for WT-control IgG, WT-Anti-C5 Ab, KO-Control IgG, KO-Anti-C5 Ab, and WT-C5a-Ant at 2 hours and 6 hours post ischemia-reperfusion.

FIG. 10 depicts Ly6G cell staining for WT-control IgG, WT-Anti-C5 Ab, KO-Control IgG, KO-Anti-C5 Ab, and WT-C5a-Ant at 6 hours post ischemia-reperfusion. Anti-C5 Ab and the WT-C5a-Ant reduced neutrophil infiltration.

FIG. 11 depicts ssDNA staining for WT-control IgG, WT-Anti-C5 Ab, KO-Control IgG, KO-Anti-C5 Ab, and WT-C5a-Ant at 6 hours post ischemia-reperfusion.

FIG. 12 depicts cleaved caspase-3 for WT Sham, WT-control IgG, WT-Anti-C5 Ab, KO Sham, KO-Control IgG, and KO-Anti-C5 Ab, 6 hours post ischemia-reperfusion, as assessed by Western Blotting.

FIG. 13 depicts cleaved caspase-3 for WT Sham, WT-control IgG, WT-Anti-C5 Ab, and WT-C5aR-Ant at 6 hours post ischemia-reperfusion, as assessed by Western Blotting.

FIG. 14 is a schematic depicting the murine model of acute/fulminant liver failure.

FIG. 15 depicts ALT release (IU/L) for WT-control IgG, WT-Anti-C5 Ab, KO-control IgG, KO-Anti-C5 Ab, and WT-C5aR Antagonist treated mice 0, 2, 4, and 6 hours after intraperitoneal administration of LPS (20 μg/kg) and D-GAIN (200 mg/kg).

FIG. 16 depicts ALT release (IU/L) for WT-control IgG, WT-Anti-C5 Ab, KO-control IgG, KO-Anti-C5 Ab, WT-C5aR Antagonist, WT sham, and KO sham treated mice 6 hours after intraperitoneal administration of LPS (20 μg/kg) and D-GAIN (200 mg/kg).

FIG. 17 depicts ALT release (IU/L) for KO- and WT-vehicle treated mice 12 hours after administration of LPS (20 μg/kg) and D-GAIN (200 mg/kg).

FIGS. 18A-18E depict levels of IL-10 (FIG. 18A), IL-6 (FIG. 18B), TNFα (FIG. 18C), CXCL-1 (FIG. 18D), and CXCL-2 (FIG. 18E) in WT-control and WT-Anti-C5 Ab treated mice.

FIG. 19 is a comparison of the injury grade of WT-control IgG, WT-Anti-C5 Ab, KO-control IgG, KO-Anti-C5 Ab, and WT-C5aR-Ant treated mice as assessed via histological analysis.

FIG. 20 depicts ssDNA staining for WT-control IgG, WT-Anti-C5 Ab, KO-control IgG, KO-Anti-C5 Ab, WT-C5aR Antagonist, WT Sham, and KO Sham treated mice at 6 hours.

FIG. 21 depicts ssDNA staining for WT-control IgG, WT-Anti-C5 Ab, KO-control IgG, KO-Anti-C5 Ab, and WT-C5aR Antagonist, at 2 hours, 4 hours, and 6 hours.

FIG. 22 depicts Ly6G staining for WT-control IgG, WT-Anti-C5 Ab, KO-control IgG, KO-Anti-C5 Ab, WT-C5aR Antagonist, WT Sham, and KO Sham treated mice at 6 hours.

FIG. 23 depicts F4/80 staining for WT-control IgG, WT-Anti-C5 Ab, KO-control IgG, KO-Anti-C5 Ab, WT-C5aR Antagonist, WT Sham, and KO Sham treated mice at 6 hours.

FIG. 24 depicts F4/80 staining for WT-control IgG, WT-Anti-C5 Ab, KO-control IgG, KO-Anti-C5 Ab, and WT-C5aR Antagonist at 2, 4, and 6 hours.

FIG. 25 depicts CD11b staining for WT-control IgG, WT-Anti-C5 Ab, KO-control IgG, KO-Anti-C5 Ab, WT-C5aR Antagonist, WT Sham, and KO Sham treated mice at 6 hours.

FIG. 26 depicts CD411 staining for WT-control IgG, WT-Anti-C5 Ab, KO-control IgG, KO-Anti-C5 Ab, WT-C5aR Antagonist, WT Sham, and KO Sham treated mice at 6 hours.

FIG. 27 depicts cleaved caspase-3/β-actin ratios for WT-sham, WT-control IgG, and WT-Anti-C5 Ab treated mice at 6 hours.

FIG. 28 depicts hemolytic activity with Zymosan for WT-control IgG and WT-Anti-C5 Ab treated mice at 2, 4, and 6 hours.

FIG. 29 depicts hemolytic activity with C5-depleted human serum for WT-control IgG and WT-Anti-C5 Ab treated mice at 2, 4, and 6 hours.

FIG. 30 depicts survival curves for KO-Anti-C5 Ab, KO-control IgG, WT-Anti-C5 Ab, and WT-control IgG treated mice over time.

FIG. 31 is a schematic depicting the murine model of acute/fulminant liver failure, wherein intravenous injection of the Anti-C5 Ab is delayed two hours after LPS/D-GaIN injection.

FIG. 32 depicts ALT release (IU/L) for WT-control IgG, WT-Anti-C5 Ab, WT-Anti-C5 Ab (administered at 2 hours), and WT-Anti-C5 Ab (administered at 4 hours) treated mice after intraperitoneal administration of LPS (20 μg/kg) and D-GAIN (200 mg/kg).

FIG. 33 is a schematic depicting the murine model to evaluate the influence of C5 blockade on liver regeneration in 70% partial hepatectomy.

FIGS. 34A-34D are graphs depicting ALT levels (FIG. 34A), CH50 levels (FIG. 34B), liver weight (FIG. 34C), and survival (FIG. 34D) at 48 hours.

FIGS. 35A and 35B are graphs depicting the percentage of BrdU-positive cells after 48 hours for the wild-type sham, wild-type control IgG, and wild-type anti-C5 antibody groups (FIG. 35A) and ALT (IU/L) versus % BRdU-positive cells after 48 hours (FIG. 35B).

### DETAILED DESCRIPTION

### I. Definitions

As used herein, the term “subject” or “patient” is a mammal (e.g., a patient (e.g., a human) having acute liver failure or who has experienced hepatic trauma due to any type of physical injury, including surgery).

As used herein, “effective treatment” refers to treatment producing a beneficial effect, e.g., amelioration of at least one symptom of a disease or disorder. A beneficial effect can take the form of an improvement over baseline, i.e., an improvement over a measurement or observation made prior to initiation of therapy according to the method. Effective treatment may refer to alleviation of at least one symptom of acute liver failure or hepatic trauma. For example, in the context of acute liver failure, effective treatment includes the alleviation of one or more symptoms selected from the group consisting of hepatic encephalopathy, impaired protein synthesis, jaundice, pain in the upper right abdomen, abdominal swelling, nausea, vomiting, malaise, disorientation, confusion, and/or sleepiness. In another embodiment, effective treatment results in a shift towards normal levels of serum albumin, a PT between 9.5 to 13.5 seconds and/or an International Normalized Ratio (INR) between 0.8 to 1.1. In the context of hepatic trauma, effective treatment can result in a decrease in one or more of the following: hepatocyte apoptosis, levels of one or more pro-inflammatory cytokines (e.g., IL-1β, IL-6, and/or TNFα) and/or one or more chemokines (e.g., CXCL-1 and/or CXCL-2), parenchymal damage markers (e.g., AST, ALT and/or T-bil), neutrophil infiltration, and/or platelet aggregation.

The term “effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In one example, an “effective amount” is the amount of an anti-C5 agent, such as an anti-C5 antibody, or antigen binding fragment thereof, clinically proven to alleviate at least one symptom of acute liver failure or hepatic trauma. An effective amount can be administered in one or more administrations.

### II. Anti-C5 Agents

An inhibitor of human complement component C5 can be, for example, a small molecule, a polypeptide, a polypeptide analog, a nucleic acid, or a nucleic acid analog.

A “small molecule” as used herein, refers to an agent, which has a molecular weight of less than about 6 kDa and most preferably less than about 2.5 kDa. Many pharmaceutical companies have extensive libraries of chemical and/or biological mixtures comprising arrays of small molecules, often fungal, bacterial, or algal extracts, which can be screened with any of the assays of the application. This application contemplates using, among other things, small chemical libraries, peptide libraries, or collections of natural products. Tan et al. described a library with over two million synthetic compounds that is compatible with miniaturized cell-based assays (J. Am. Chem. Soc. (1998) 120:8565-8566). It is within the scope of this application that such a library may be used to screen for inhibitors of human complement component C5. There are numerous commercially available compound libraries, such as the Chembridge DIVERSet. Libraries are also available from academic investigators, such as the Diversity set from the NCI developmental therapeutics program. Rational drug design may also be employed. For example, rational drug design can employ the use of crystal or solution structural information on the human complement component C5 protein. See, e.g., the structures described in Hagemann et al. (2008) J Biol Chem 283(12):7763-75 and Zuiderweg et al. (1989) Biochemistry 28(1):172-85. Rational drug design can also be achieved based on known compounds, e.g., a known inhibitor of C5 (e.g., an antibody, or antigen-binding fragment thereof, that binds to a human complement component C5 protein).

Peptidomimetics are compounds in which at least a portion of a subject polypeptide is modified, and the three-dimensional structure of the peptidomimetic remains substantially the same as that of the subject polypeptide. Peptidomimetics may be analogues of a subject polypeptide of the disclosure that are, themselves, polypeptides containing one or more substitutions or other modifications within the subject polypeptide sequence. Alternatively, at least a portion of the subject polypeptide sequence may be replaced with a non-peptide structure, such that the three-dimensional structure of the subject polypeptide is substantially retained. In other words, one, two or three amino acid residues within the subject polypeptide sequence may be replaced by a non-peptide structure. In addition, other peptide portions of the subject polypeptide may, but need not, be replaced with a non-peptide structure. Peptidomimetics (both peptide and non-peptidyl analogues) may have improved properties (e.g., decreased proteolysis, increased retention or increased bioavailability). Peptidomimetics generally have improved oral availability, which makes them especially suited to treatment of disorders in a human or animal. It should be noted that peptidomimetics may or may not have similar two-dimensional chemical structures, but share common three-dimensional structural features and geometry. Each peptidomimetic may further have one or more unique additional binding elements.

Nucleic acid inhibitors can be used to decrease expression of an endogenous gene encoding human complement component C5. The nucleic acid antagonist can be, e.g., an siRNA, a dsRNA, a ribozyme, a triple-helix former, an aptamer, or an antisense nucleic acid. siRNAs are small double stranded RNAs (dsRNAs) that optionally include overhangs. For example, the duplex region of an siRNA is about 18 to 25 nucleotides in length, e.g., about 19, 20, 21, 22, 23, or 24 nucleotides in length. The siRNA sequences can be, in some embodiments, exactly complementary to the target mRNA. dsRNAs and siRNAs, in particular, can be used to silence gene expression in mammalian cells (e.g., human cells). See, e.g., Clemens et al. (2000) Proc. Natl. Acad. Sci. USA 97:6499-6503; Billy et al. (2001) Proc. Natl. Acad. Sci. USA 98:14428-14433; Elbashir et al. (2001) Nature 411:494-8; Yang et al. (2002) Proc. Natl. Acad. Sci. USA 99:9942-9947, and U.S. Patent Application Publication Nos. 20030166282, 20030143204, 20040038278, and 20030224432. Anti-sense agents can include, for example, from about 8 to about 80 nucleobases (i.e. from about 8 to about 80 nucleotides), e.g., about 8 to about 50 nucleobases, or about 12 to about 30 nucleobases. Anti-sense compounds include ribozymes, external guide sequence (EGS) oligonucleotides (oligozymes), and other short catalytic RNAs or catalytic oligonucleotides which hybridize to the target nucleic acid and modulate its expression. Anti-sense compounds can include a stretch of at least eight consecutive nucleobases that are complementary to a sequence in the target gene. An oligonucleotide need not be 100% complementary to its target nucleic acid sequence to be specifically hybridizable. An oligonucleotide is specifically hybridizable when binding of the oligonucleotide to the target interferes with the normal function of the target molecule to cause a loss of utility, and there is a sufficient degree of complementarity to avoid non-specific binding of the oligonucleotide to non-target sequences under conditions in which specific binding is desired, i.e., under physiological conditions in the case of in vivo assays or therapeutic treatment or, in the case of in vitro assays, under conditions in which the assays are conducted. Hybridization of antisense oligonucleotides with mRNA (e.g., an mRNA encoding a human C5 protein) can interfere with one or more of the normal functions of mRNA. The functions of mRNA to be interfered with include all key functions such as, for example, translocation of the RNA to the site of protein translation, translation of protein from the RNA, splicing of the RNA to yield one or more mRNA species, and catalytic activity which may be engaged in by the RNA. Binding of specific protein(s) to the RNA may also be interfered with by antisense oligonucleotide hybridization to the RNA. Exemplary antisense compounds include DNA or RNA sequences that specifically hybridize to the target nucleic acid, e.g., the mRNA encoding a human complement component C5 protein. The complementary region can extend for between about 8 to about 80 nucleobases. The compounds can include one or more modified nucleobases.

Modified nucleobases may include, e.g., 5-substituted pyrimidines such as 5-iodouracil, 5-iodocytosine, and C.sub.5-propynyl pyrimidines such as C.sub.5-propynylcytosine and C.sub.5-propynyluracil. Other suitable modified nucleobases include, e.g., 7-substituted-8-aza-7-deazapurines and 7-substituted-7-deazapurines such as, for example, 7-iodo-7-deazapurines, 7-cyano-7-deazapurines, 7-aminocarbonyl-7-deazapurines. Examples of these include 6-amino-7-iodo-7-deazapurines, 6-amino-7-cyano-7-deazapurines, 6-amino-7-aminocarbonyl-7-deazapurines, 2-amino-6-hydroxy-7-iodo-7-deazapurines, 2-amino-6-hydroxy-7-cyano-7-deazapurines, and 2-amino-6-hydroxy-7-aminocarbonyl-7-deazapurines. See, e.g., U.S. Pat. Nos. 4,987,071; 5,116,742; and U.S. Pat. No. 5,093,246; “Antisense RNA and DNA,” D. A. Melton, Ed., Cold Spring Harbor Laboratory, Cold Spring Harbor, N.Y. (1988); Haselhoff and Gerlach (1988) Nature 334:585-59; Helene, C. (1991) Anticancer Drug D 6:569-84; Helene (1992) Ann. NY. Acad. Sci. 660:27-36; and Maher (1992) Bioassays 14:807-15.

Aptamers are short oligonucleotide sequences that can be used to recognize and specifically bind almost any molecule, including cell surface proteins. The systematic evolution of ligands by exponential enrichment (SELEX) process is powerful and can be used to readily identify such aptamers. Aptamers can be made for a wide range of proteins of importance for therapy and diagnostics, such as growth factors and cell surface antigens. These oligonucleotides bind their targets with similar affinities and specificities as antibodies do (see, e.g., Ulrich (2006) Handb Exp Pharmacol. 173:305-326).

### III. Anti-C5 Antibodies

The anti-C5 antibodies described herein bind to complement component C5 (e.g., human C5) and inhibit the cleavage of C5 into fragments C5a and C5b. Anti-C5 antibodies (or VH/VL domains derived therefrom) suitable for use in the invention can be generated using methods well known in the art. Alternatively, art recognized anti-C5 antibodies can be used. Antibodies or any other agents that compete with any of these art-recognized antibodies for binding to C5 also can be used.

The term “antibody” describes polypeptides comprising at least one antibody derived antigen binding site (e.g., VH/VL region or Fv, or CDR). Antibodies include known forms of antibodies. For example, the antibody can be a human antibody, a humanized antibody, a bispecific antibody, or a chimeric antibody. The antibody also can be a Fab, Fab′2, ScFv, SMIP, Affibody®, nanobody, or a domain antibody. The antibody also can be of any of the following isotypes: IgG1, IgG2, IgG3, IgG4, IgM, IgA1, IgA2, IgAsec, IgD, and IgE. The antibody may be a naturally occurring antibody or may be an antibody that has been altered by a protein engineering technique (e.g., by mutation, deletion, substitution, conjugation to a non-antibody moiety). For example, an antibody may include one or more variant amino acids (compared to a naturally occurring antibody), which changes a property (e.g., a functional property) of the antibody. For example, numerous such alterations are known in the art which affect, e.g., half-life, effector function, and/or immune responses to the antibody in a patient. The term antibody also includes artificial or engineered polypeptide constructs which comprise at least one antibody-derived antigen binding site.

Eculizumab (also known as Soliris®) is an anti-C5 antibody comprising heavy and light chains having sequences shown in SEQ ID NO: 10 and 11, respectively, or antigen binding fragments and variants thereof. The variable regions of eculizumab are described in PCT/US1995/005688 and U.S. Pat. No. 6,355,245, the teachings of which are hereby incorporated by reference. The full heavy and light chains of eculizumab are described in PCT/US2007/006606, the teachings of which are hereby incorporated by reference. In one embodiment the anti-C5 antibody, comprises the CDR1, CDR2, and CDR3 domains of the VH region of eculizumab having the sequence set forth in SEQ ID NO: 7, and the CDR1, CDR2 and CDR3 domains of the VL region of eculizumab having the sequence set forth in SEQ ID NO: 8. In another embodiment, the antibody comprises heavy chain CDR1, CDR2 and CDR3 domains having the sequences set forth in SEQ ID NOs: 1, 2, and 3, respectively, and light chain CDR1, CDR2 and CDR3 domains having the sequences set forth in SEQ ID NOs: 4, 5, and 6, respectively. In another embodiment, the antibody comprises VH and VL regions having the amino acid sequences set forth in SEQ ID NO: 7 and SEQ ID NO: 8, respectively.

An exemplary anti-C5 antibody is ravulizumab comprising heavy and light chains having the sequences shown in SEQ ID NOs:14 and 11, respectively, or antigen binding fragments and variants thereof. Ravulizumab (also known as BNJ441 and ALXN1210) is described in PCT/US2015/019225 and U.S. Pat. No. 9,079,949, the teachings of which are hereby incorporated by reference. The terms ravulizumab, BNJ441, and ALXN1210 may be used interchangeably throughout this document, but all refer to the same antibody. Ravulizumab selectively binds to human complement protein C5, inhibiting its cleavage to C5a and C5b during complement activation. This inhibition prevents the release of the proinflammatory mediator C5a and the formation of the cytolytic pore-forming membrane attack complex (MAC) C5b-9 while preserving the proximal or early components of complement activation (e.g., C3 and C3b) essential for the opsonization of microorganisms and clearance of immune complexes.

In other embodiments, the antibody comprises the heavy and light chain CDRs or variable regions of ravulizumab. For example, in one embodiment, the antibody comprises the CDR1, CDR2, and CDR3 domains of the VH region of ravulizumab having the sequence set forth in SEQ ID NO:12, and the CDR1, CDR2 and CDR3 domains of the VL region of ravulizumab having the sequence set forth in SEQ ID NO:8. In another embodiment, the antibody comprises heavy chain CDR1, CDR2 and CDR3 domains having the sequences set forth in SEQ ID NOs:19, 18, and 3, respectively, and light chain CDR1, CDR2 and CDR3 domains having the sequences set forth in SEQ ID NOs:4, 5, and 6, respectively. In another embodiment, the antibody comprises VH and VL regions having the amino acid sequences set forth in SEQ ID NO:12 and SEQ ID NO:8, respectively.

Another exemplary anti-C5 antibody is antibody BNJ421 comprising heavy and light chains having the sequences shown in SEQ ID NOs:20 and 11, respectively, or antigen binding fragments and variants thereof. BNJ421 (also known as ALXN1211) is described in PCT/US2015/019225 and U.S. Pat. No. 9,079,949, the teachings or which are hereby incorporated by reference.

In other embodiments, the antibody comprises the heavy and light chain CDRs or variable regions of BNJ421. Accordingly, in one embodiment, the antibody comprises the CDR1, CDR2, and CDR3 domains of the VH region of BNJ421 having the sequence set forth in SEQ ID NO:12, and the CDR1, CDR2 and CDR3 domains of the VL region of BNJ421 having the sequence set forth in SEQ ID NO:8. In another embodiment, the antibody comprises heavy chain CDR1, CDR2 and CDR3 domains having the sequences set forth in SEQ ID NOs:19, 18, and 3, respectively, and light chain CDR1, CDR2 and CDR3 domains having the sequences set forth in SEQ ID NOs:4, 5, and 6, respectively. In another embodiment, the antibody comprises VH and VL regions having the amino acid sequences set forth in SEQ ID NO:12 and SEQ ID NO:8, respectively.

The exact boundaries of CDRs have been defined differently according to different methods. In some embodiments, the positions of the CDRs or framework regions within a light or heavy chain variable domain can be as defined by Kabat et al. [(1991) “Sequences of Proteins of Immunological Interest.” NIH Publication No. 91-3242, U.S. Department of Health and Human Services, Bethesda, Md.]. In such cases, the CDRs can be referred to as “Kabat CDRs” (e.g., “Kabat LCDR2” or “Kabat HCDR1”). In some embodiments, the positions of the CDRs of a light or heavy chain variable region can be as defined by Chothia et al. (1989) Nature 342:877-883. Accordingly, these regions can be referred to as “Chothia CDRs” (e.g., “Chothia LCDR2” or “Chothia HCDR3”). In some embodiments, the positions of the CDRs of the light and heavy chain variable regions can be as defined by a Kabat-Chothia combined definition. In such embodiments, these regions can be referred to as “combined Kabat-Chothia CDRs”. Thomas et al. [(1996) Mol Immunol 33(17/18):1389-1401] exemplifies the identification of CDR boundaries according to Kabat and Chothia definitions.

In some embodiments, an anti-C5 antibody described herein comprises a heavy chain CDR1 comprising, or consisting of, the following amino acid sequence: GHIFSNYWIQ (SEQ ID NO:19). In some embodiments, an anti-C5 antibody described herein comprises a heavy chain CDR2 comprising, or consisting of, the following amino acid sequence: EILPGSGHTEYTENFKD (SEQ ID NO:18). In some embodiments, an anti-C5 antibody described herein comprises a heavy chain variable region comprising the following amino acid sequence:

(SEQ ID NO: 12)
QVQLVQSGAEVKKPGASVKVSCKASGHIFSNYWIQWVRQAPGQGLEWMGE
ILPGSGHTEYTENFKDRVTMTRDTSTSTVYMELSSLRSEDTAVYYCARYF
FGSSPNWYFDVWGQGTLVTVSS.

In some embodiments, an anti-C5 antibody described herein comprises a light chain variable region comprising the following amino acid sequence:

(SEQ ID NO: 8)
DIQMTQSPSSLSASVGDRVTITCGASENIYGALNWYQQKPGKAPKLLIYG
ATNLADGVPSRFSGSGSGTDFTLTISSLQPEDFATYYCQNVLNTPLTFGQ
GTKVEIK.

Another exemplary anti-C5 antibody is the 7086 antibody described in U.S. Pat. Nos. 8,241,628 and 8,883,158. In one embodiment, the antibody comprises the heavy and light chain CDRs or variable regions of the 7086 antibody (see U.S. Pat. Nos. 8,241,628 and 8,883,158). In another embodiment, the antibody, or antigen binding fragment thereof, comprises heavy chain CDR1, CDR2 and CDR3 domains having the sequences set forth in SEQ ID NOs: 21, 22, and 23, respectively, and light chain CDR1, CDR2 and CDR3 domains having the sequences set forth in SEQ ID NOs: 24, 25, and 26, respectively. In another embodiment, the antibody, or antigen binding fragment thereof, comprises the VH region of the 7086 antibody having the sequence set forth in SEQ ID NO:27, and the VL region of the 7086 antibody having the sequence set forth in SEQ ID NO:28.

Another exemplary anti-C5 antibody is the 8110 antibody also described in U.S. Pat. Nos. 8,241,628 and 8,883,158. In one embodiment, the antibody comprises the heavy and light chain CDRs or variable regions of the 8110 antibody. In another embodiment, the antibody, or antigen binding fragment thereof, comprises heavy chain CDR1, CDR2 and CDR3 domains having the sequences set forth in SEQ ID NOs: 29, 30, and 31, respectively, and light chain CDR1, CDR2 and CDR3 domains having the sequences set forth in SEQ ID NOs: 32, 33, and 34, respectively. In another embodiment, the antibody comprises the VH region of the 8110 antibody having the sequence set forth in SEQ ID NO: 35, and the VL region of the 8110 antibody having the sequence set forth in SEQ ID NO: 36.

Another exemplary anti-C5 antibody is the 305L05 antibody described in US2016/0176954A1. In one embodiment, the antibody comprises the heavy and light chain CDRs or variable regions of the 305L05 antibody. In another embodiment, the antibody, or antigen binding fragment thereof, comprises heavy chain CDR1, CDR2 and CDR3 domains having the sequences set forth in SEQ ID NOs: 37, 38, and 39, respectively, and light chain CDR1, CDR2 and CDR3 domains having the sequences set forth in SEQ ID NOs: 40, 41, and 42, respectively. In another embodiment, the antibody comprises the VH region of the 305L05 antibody having the sequence set forth in SEQ ID NO: 43, and the VL region of the 305L05 antibody having the sequence set forth in SEQ ID NO: 44.

Another exemplary anti-C5 antibody is the SKY59 antibody described in Fukuzawa T., et al., Rep. 2017 Apr. 24; 7(1):1080). In one embodiment, the antibody comprises the heavy and light chain CDRs or variable regions of the SKY59 antibody. In another embodiment, the antibody, or antigen binding fragment thereof, comprises a heavy chain comprising SEQ ID NO: 45 and a light chain comprising SEQ ID NO: 46.

Another exemplary anti-C5 antibody is the REGN3918 antibody (also known as H4H12166PP) described in US20170355757. In one embodiment, the antibody comprises a heavy chain variable region comprising SEQ ID NO:47 and a light chain variable region comprising SEQ ID NO:48. In another embodiment, the antibody comprises a heavy chain comprising SEQ ID NO:49 and a light chain comprising SEQ ID NO:50.

An anti-C5 antibody described herein can, in some embodiments, comprise a variant human Fc constant region that binds to human neonatal Fc receptor (FcRn) with greater affinity than that of the native human Fc constant region from which the variant human Fc constant region was derived. For example, the Fc constant region can comprise one or more (e.g., two, three, four, five, six, seven, or eight or more) amino acid substitutions relative to the native human Fc constant region from which the variant human Fc constant region was derived. The substitutions can increase the binding affinity of an IgG antibody containing the variant Fc constant region to FcRn at pH 6.0, while maintaining the pH dependence of the interaction. Methods for testing whether one or more substitutions in the Fc constant region of an antibody increase the affinity of the Fc constant region for FcRn at pH 6.0 (while maintaining pH dependence of the interaction) are known in the art.

Substitutions that enhance the binding affinity of an antibody Fc constant region for FcRn are known in the art and include, e.g., (1) the M252Y/S254T/T256E triple substitution described by Dall'Acqua et al. (2006) J Biol Chem 281: 23514-23524; (2) the M428L or T250Q/M428L substitutions described in Hinton et al. (2004) J Biol Chem 279:6213-6216 and Hinton et al. (2006) J Immunol 176:346-356; and (3) the N434A or T307/E380A/N434A substitutions described in Petkova et al. (2006) Int Immunol 18(12):1759-69. The additional substitution pairings: P257I/Q3111, P257I/N434H, and D376V/N434H are described in, e.g., Datta-Mannan et al. (2007) J Biol Chem 282(3):1709-1717, the disclosure of which is incorporated herein by reference in its entirety.

In some embodiments, the variant constant region has a substitution at EU amino acid residue 255 for valine. In some embodiments, the variant constant region has a substitution at EU amino acid residue 309 for asparagine. In some embodiments, the variant constant region has a substitution at EU amino acid residue 312 for isoleucine. In some embodiments, the variant constant region has a substitution at EU amino acid residue 386.

In some embodiments, the variant Fc constant region comprises no more than 30 (e.g., no more than 29, 28, 27, 26, 25, 24, 23, 22, 21, 20, 19, 18, 17, 16, 15, 14, 13, 12, 11, 10, nine, eight, seven, six, five, four, three, or two) amino acid substitutions, insertions, or deletions relative to the native constant region from which it was derived. In some embodiments, the variant Fc constant region comprises one or more amino acid substitutions selected from the group consisting of: M252Y, S254T, T256E, N434S, M428L, V259I, T250I, and V308F. In some embodiments, the variant human Fc constant region comprises a methionine at position 428 and an asparagine at position 434 of a native human IgG Fc constant region, each in EU numbering. In some embodiments, the variant Fc constant region comprises a 428L/434S double substitution as described in, e.g., U.S. Pat. No. 8,088,376.

In some embodiments the precise location of these mutations may be shifted from the native human Fc constant region position due to antibody engineering. For example, the 428L/434S double substitution when used in a IgG2/4 chimeric Fc may correspond to 429L and 435S as in the M429L and N435S variants found in BNJ441 (ravulizumab) and described in U.S. Pat. No. 9,079,949 the disclosure of which is incorporated herein by reference in its entirety.

In some embodiments, the variant constant region comprises a substitution at amino acid position 237, 238, 239, 248, 250, 252, 254, 255, 256, 257, 258, 265, 270, 286, 289, 297, 298, 303, 305, 307, 308, 309, 311, 312, 314, 315, 317, 325, 332, 334, 360, 376, 380, 382, 384, 385, 386, 387, 389, 424, 428, 433, 434, or 436 (EU numbering) relative to the native human Fc constant region. In some embodiments, the substitution is selected from the group consisting of: methionine for glycine at position 237; alanine for proline at position 238; lysine for serine at position 239; isoleucine for lysine at position 248; alanine, phenylalanine, isoleucine, methionine, glutamine, serine, valine, tryptophan, or tyrosine for threonine at position 250; phenylalanine, tryptophan, or tyrosine for methionine at position 252; threonine for serine at position 254; glutamic acid for arginine at position 255; aspartic acid, glutamic acid, or glutamine for threonine at position 256; alanine, glycine, isoleucine, leucine, methionine, asparagine, serine, threonine, or valine for proline at position 257; histidine for glutamic acid at position 258; alanine for aspartic acid at position 265; phenylalanine for aspartic acid at position 270; alanine, or glutamic acid for asparagine at position 286; histidine for threonine at position 289; alanine for asparagine at position 297; glycine for serine at position 298; alanine for valine at position 303; alanine for valine at position 305; alanine, aspartic acid, phenylalanine, glycine, histidine, isoleucine, lysine, leucine, methionine, asparagine, proline, glutamine, arginine, serine, valine, tryptophan, or tyrosine for threonine at position 307; alanine, phenylalanine, isoleucine, leucine, methionine, proline, glutamine, or threonine for valine at position 308; alanine, aspartic acid, glutamic acid, proline, or arginine for leucine or valine at position 309; alanine, histidine, or isoleucine for glutamine at position 311; alanine or histidine for aspartic acid at position 312; lysine or arginine for leucine at position 314; alanine or histidine for asparagine at position 315; alanine for lysine at position 317; glycine for asparagine at position 325; valine for isoleucine at position 332; leucine for lysine at position 334; histidine for lysine at position 360; alanine for aspartic acid at position 376; alanine for glutamic acid at position 380; alanine for glutamic acid at position 382; alanine for asparagine or serine at position 384; aspartic acid or histidine for glycine at position 385; proline for glutamine at position 386; glutamic acid for proline at position 387; alanine or serine for asparagine at position 389; alanine for serine at position 424; alanine, aspartic acid, phenylalanine, glycine, histidine, isoleucine, lysine, leucine, asparagine, proline, glutamine, serine, threonine, valine, tryptophan, or tyrosine for methionine at position 428; lysine for histidine at position 433; alanine, phenylalanine, histidine, serine, tryptophan, or tyrosine for asparagine at position 434; and histidine for tyrosine or phenylalanine at position 436, all in EU numbering.

Suitable anti-C5 antibodies for use in the methods described herein, in some embodiments, comprise a heavy chain polypeptide comprising the amino acid sequence depicted in SEQ ID NO:14 and/or a light chain polypeptide comprising the amino acid sequence depicted in SEQ ID NO:11. Alternatively, the anti-C5 antibodies for use in the methods described herein, in some embodiments, comprise a heavy chain polypeptide comprising the amino acid sequence depicted in SEQ ID NO:20 and/or a light chain polypeptide comprising the amino acid sequence depicted in SEQ ID NO:11.

In one embodiment, the antibody binds to C5 at pH 7.4 and 25° C. (and, otherwise, under physiologic conditions) with an affinity dissociation constant (K_{D}) that is at least 0.1 (e.g., at least 0.15, 0.175, 0.2, 0.25, 0.275, 0.3, 0.325, 0.35, 0.375, 0.4, 0.425, 0.45, 0.475, 0.5, 0.525, 0.55, 0.575, 0.6, 0.625, 0.65, 0.675, 0.7, 0.725, 0.75, 0.775, 0.8, 0.825, 0.85, 0.875, 0.9, 0.925, 0.95, or 0.975) nM. In some embodiments, the K_{D} of the anti-C5 antibody, or antigen binding fragment thereof, is no greater than 1 (e.g., no greater than 0.9, 0.8, 0.7, 0.6, 0.5, 0.4, 0.3, or 0.2) nM.

In other embodiments, the [(K_{D} of the antibody for C5 at pH 6.0 at C)/(K_{D} of the antibody for C5 at pH 7.4 at 25° C.)] is greater than 21 (e.g., greater than 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 350, 400, 450, 500, 600, 700, 800, 900, 1000, 1500, 2000, 2500, 3000, 3500, 4000, 4500, 5000, 5500, 6000, 6500, 7000, 7500, or 8000).

Methods for determining whether an antibody binds to a protein antigen and/or the affinity for an antibody to a protein antigen are known in the art. For example, the binding of an antibody to a protein antigen can be detected and/or quantified using a variety of techniques such as, but not limited to, Western blot, dot blot, surface plasmon resonance (SPR) method (e.g., BIAcore system; Pharmacia Biosensor AB, Uppsala, Sweden and Piscataway, N.J.), or enzyme-linked immunosorbent assay (ELISA). See, e.g., Benny K. C. Lo (2004) “Antibody Engineering: Methods and Protocols,” Humana Press (ISBN: 1588290921); Johne et al. (1993) J Immunol Meth 160:191-198; Jonsson et al. (1993) Ann Biol Clin 51:19-26; and Jonsson et al. (1991) Biotechniques 11:620-627. In addition, methods for measuring the affinity (e.g., dissociation and association constants) are set forth in the working examples.

As used herein, the term “kₐ” refers to the rate constant for association of an antibody to an antigen. The term “k_{d}” refers to the rate constant for dissociation of an antibody from the antibody/antigen complex. And the term “K_{D}” refers to the equilibrium dissociation constant of an antibody-antigen interaction. The equilibrium dissociation constant is deduced from the ratio of the kinetic rate constants, K_{D}=kₐ/k_{d}. Such determinations preferably are measured at 25° C. or 37° C. (see the working examples). For example, the kinetics of antibody binding to human C5 can be determined at pH 8.0, 7.4, 7.0, 6.5 and 6.0 via surface plasmon resonance (SPR) on a BIAcore 3000 instrument using an anti-Fc capture method to immobilize the antibody.

In one embodiment, the anti-C5 antibody, or antigen binding fragment thereof, blocks the generation or activity of the C5a and/or C5b active fragments of a C5 protein (e.g., a human C5 protein). Through this blocking effect, the antibodies inhibit, e.g., the pro-inflammatory effects of C5a and the generation of the C5b-9 membrane attack complex (MAC) at the surface of a cell.

Methods for determining whether a particular antibody or therapeutic agent described herein inhibits C5 cleavage are known in the art. Inhibition of human complement component C5 can reduce the cell-lysing ability of complement in a subject's body fluids. Such reductions of the cell-lysing ability of complement present in the body fluid(s) can be measured by methods well known in the art such as, for example, by a conventional hemolytic assay such as the hemolysis assay described by Kabat and Mayer (eds.), “Experimental Immunochemistry, 2^{nd} Edition,” 135-240, Springfield, Ill., CC Thomas (1961), pages 135-139, or a conventional variation of that assay such as the chicken erythrocyte hemolysis method as described in, e.g., Hillmen et al. (2004) N Engl J Med 350(6):552. Methods for determining whether a candidate compound inhibits the cleavage of human C5 into forms C5a and C5b are known in the art and described in Evans et al. (1995) Mol Immunol 32(16):1183-95. For example, the concentration and/or physiologic activity of C5a and C5b in a body fluid can be measured by methods well known in the art. For C5b, hemolytic assays or assays for soluble C5b-9 as discussed herein can be used. Other assays known in the art can also be used. Using assays of these or other suitable types, candidate agents capable of inhibiting human complement component C5 can be screened.

Immunological techniques such as, but not limited to, ELISA can be used to measure the protein concentration of C5 and/or its split products to determine the ability of an anti-C5 antibody, or antigen binding fragment thereof, to inhibit conversion of C5 into biologically active products. In some embodiments, C5a generation is measured. In some embodiments, C5b-9 neoepitope-specific antibodies are used to detect the formation of terminal complement.

Hemolytic assays can be used to determine the inhibitory activity of an anti-C5 antibody, or antigen binding fragment thereof, on complement activation. In order to determine the effect of an anti-C5 antibody, or antigen binding fragment thereof, on classical complement pathway-mediated hemolysis in a serum test solution in vitro, for example, sheep erythrocytes coated with hemolysin or chicken erythrocytes sensitized with anti-chicken erythrocyte antibody are used as target cells. The percentage of lysis is normalized by considering 100% lysis equal to the lysis occurring in the absence of the inhibitor. In some embodiments, the classical complement pathway is activated by a human IgM antibody, for example, as utilized in the Wieslab® Classical Pathway Complement Kit (Wieslab® COMPL CP310, Euro-Diagnostica, Sweden). Briefly, the test serum is incubated with an anti-C5 antibody, or antigen binding fragment thereof, in the presence of a human IgM antibody. The amount of C5b-9 that is generated is measured by contacting the mixture with an enzyme conjugated anti-C5b-9 antibody and a fluorogenic substrate and measuring the absorbance at the appropriate wavelength. As a control, the test serum is incubated in the absence of the anti-C5 antibody, or antigen binding fragment thereof. In some embodiments, the test serum is a C5-deficient serum reconstituted with a C5 polypeptide.

To determine the effect of an anti-C5 antibody, or antigen binding fragment thereof, on alternative pathway-mediated hemolysis, unsensitized rabbit or guinea pig erythrocytes can be used as the target cells. In some embodiments, the serum test solution is a C5-deficient serum reconstituted with a C5 polypeptide. The percentage of lysis is normalized by considering 100% lysis equal to the lysis occurring in the absence of the inhibitor. In some embodiments, the alternative complement pathway is activated by lipopolysaccharide molecules, for example, as utilized in the Wieslab® Alternative Pathway Complement Kit (Wieslab® COMPL AP330, Euro-Diagnostica, Sweden). Briefly, the test serum is incubated with an anti-C5 antibody, or antigen binding fragment thereof, in the presence of lipopolysaccharide. The amount of C5b-9 that is generated is measured by contacting the mixture with an enzyme conjugated anti-C5b-9 antibody and a fluorogenic substrate and measuring the fluorescence at the appropriate wavelength. As a control, the test serum is incubated in the absence of the anti-C5 antibody, or antigen binding fragment thereof.

In some embodiments, C5 activity, or inhibition thereof, is quantified using a CH50eq assay. The CH50eq assay is a method for measuring the total classical complement activity in serum. This test is a lytic assay, which uses antibody-sensitized erythrocytes as the activator of the classical complement pathway and various dilutions of the test serum to determine the amount required to give 50% lysis (CH50). The percent hemolysis can be determined, for example, using a spectrophotometer. The CH50eq assay provides an indirect measure of terminal complement complex (TCC) formation, since the TCC themselves are directly responsible for the hemolysis that is measured.

The assay is well known and commonly practiced by those of skill in the art. Briefly, to activate the classical complement pathway, undiluted serum samples (e.g., reconstituted human serum samples) are added to microassay wells containing the antibody-sensitized erythrocytes to thereby generate TCC. Next, the activated sera are diluted in microassay wells, which are coated with a capture reagent (e.g., an antibody that binds to one or more components of the TCC). The TCC present in the activated samples bind to the monoclonal antibodies coating the surface of the microassay wells. The wells are washed and to each well is added a detection reagent that is detectably labeled and recognizes the bound TCC. The detectable label can be, e.g., a fluorescent label or an enzymatic label. The assay results are expressed in CH50 unit equivalents per milliliter (CH50 U Eq/mL).

Inhibition, e.g., as it pertains to terminal complement activity, includes at least a 5 (e.g., at least a 6, 7, 8, 9, 10, 15, 20, 25, 30, 35, 40, 45, 50, 55, or 60) % decrease in the activity of terminal complement in, e.g., a hemolytic assay or CH50eq assay as compared to the effect of a control antibody (or antigen-binding fragment thereof) under similar conditions and at an equimolar concentration. Substantial inhibition, as used herein, refers to inhibition of a given activity (e.g., terminal complement activity) of at least 40 (e.g., at least 45, 50, 55, 60, 65, 70, 75, 80, 85, 90, or 95 or greater) %. In some embodiments, an anti-C5 antibody described herein contains one or more amino acid substitutions relative to the CDRs of eculizumab (i.e., SEQ ID NOs:1-6), yet retains at least 30 (e.g., at least 31, 32, 33, 34, 35, 36, 37, 38, 39, 40, 41, 42, 43, 44, 45, 46, 47, 48, 49, 50, 55, 60, 65, 70, 75, 80, 85, 90, or 95) % of the complement inhibitory activity of eculizumab in a hemolytic assay or CH50eq assay.

In one embodiment, the antibody competes for binding with, and/or binds to the same epitope on C5 as, the antibodies described herein. The term “binds to the same epitope” with reference to two or more antibodies means that the antibodies bind to the same segment of amino acid residues, as determined by a given method. Techniques for determining whether antibodies bind to the “same epitope on C5” with the antibodies described herein include, for example, epitope mapping methods, such as, x-ray analyses of crystals of antigen:antibody complexes which provides atomic resolution of the epitope and hydrogen/deuterium exchange mass spectrometry (HDX-MS). Other methods monitor the binding of the antibody to peptide antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. Antibodies having the same VH and VL or the same CDR1, 2 and 3 sequences are expected to bind to the same epitope.

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, may be determined using known competition experiments. In certain embodiments, an antibody competes with, and inhibits binding of another antibody to a target by at least 10%, 20%, 30%, 40%, 50%, 60%, 70%, 80%, 90% or 100%. The level of inhibition or competition may be different depending on which antibody is the “blocking antibody” (i.e., the cold antibody that is incubated first with the target). Competing antibodies bind to the same epitope, an overlapping epitope or to adjacent epitopes (e.g., as evidenced by steric hindrance).

Anti-C5 antibodies, or antigen-binding fragments thereof described herein, used in the methods described herein can be generated using a variety of art-recognized techniques. Monoclonal antibodies may be obtained by various techniques familiar to those skilled in the art. Briefly, spleen cells from an animal immunized with a desired antigen are immortalized, commonly by fusion with a myeloma cell (see, Kohler & Milstein, Eur. J. Immunol. 6: 511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse, et al., Science 246: 1275-1281 (1989).

### IV. Compositions and Administration

Also, provided herein are compositions (e.g., formulations) comprising an anti-C5 agent, such as an antibody, or antigen binding fragment thereof for use in the methods described herein. The compositions can be formulated as a pharmaceutical solution for administration to a subject. The pharmaceutical compositions will generally include a pharmaceutically acceptable carrier. As used herein, a “pharmaceutically acceptable carrier” refers to, and includes, any and all solvents, dispersion media, coatings, antibacterial and antifungal agents, isotonic and absorption delaying agents, and the like that are physiologically compatible. The compositions can include a pharmaceutically acceptable salt, e.g., an acid addition salt or a base addition salt, sugars, carbohydrates, polyols and/or tonicity modifiers.

The compositions can be formulated according to standard methods. Pharmaceutical formulation is a well-established art, and is further described in, e.g., Gennaro (2000) “Remington: The Science and Practice of Pharmacy,” 20^{th} Edition, Lippincott, Williams & Wilkins (ISBN: 0683306472); Ansel et al. (1999) “Pharmaceutical Dosage Forms and Drug Delivery Systems,” 7^{th} Edition, Lippincott Williams & Wilkins Publishers (ISBN: 0683305727); and Kibbe (2000) “Handbook of Pharmaceutical Excipients American Pharmaceutical Association,” 3^{rd} Edition (ISBN: 091733096X). In some embodiments, a composition can be formulated, for example, as a buffered solution at a suitable concentration and suitable for storage at 2-8° C. (e.g., 4° C.). In some embodiments, a composition can be formulated for storage at a temperature below 0° C. (e.g., −20° C. or −80° C.). In some embodiments, the composition can be formulated for storage for up to 2 years (e.g., one month, two months, three months, four months, five months, six months, seven months, eight months, nine months, 10 months, 11 months, 1 year, 1½ years, or 2 years) at 2-8° C. (e.g., 4° C.). Thus, in some embodiments, the compositions described herein are stable in storage for at least 1 year at 2-8° C. (e.g., 4° C.).

The pharmaceutical compositions can be in a variety of forms. These forms include, e.g., liquid, semi-solid and solid dosage forms, such as liquid solutions (e.g., injectable and infusible solutions), dispersions or suspensions, tablets, pills, powders, liposomes and suppositories. The preferred form depends, in part, on the intended mode of administration and therapeutic application. For example, compositions containing a composition intended for systemic or local delivery can be in the form of injectable or infusible solutions. Accordingly, the compositions can be formulated for administration by a parenteral mode (e.g., intravenous, subcutaneous, intraperitoneal, or intramuscular injection). “Parenteral administration,” “administered parenterally,” and other grammatically equivalent phrases, as used herein, refer to modes of administration other than enteral and topical administration, usually by injection, and include, without limitation, intravenous, intranasal, intraocular, pulmonary, intramuscular, intraarterial, intrathecal, intracapsular, intraorbital, intracardiac, intradermal, intrapulmonary, intraperitoneal, transtracheal, subcutaneous, subcuticular, intraarticular, subcapsular, subarachnoid, intraspinal, epidural, intracerebral, intracranial, intracarotid and intrasternal injection and infusion.

The anti-C5 agent (e.g., antibody, or antigen binding fragment thereof), can be administered to a patient by any suitable means. In one embodiment, the anti-C5 agent (e.g., anti-C5 antibody, or antigen binding fragment thereof), is administered intravenously. In another embodiment, the anti-C5 agent is an antibody, or antigen binding fragment thereof, administered at a dose of about 400 mg, 405 mg, 410 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, 800 mg, 805 mg, 810 mg, 815 mg, 820 mg, 825 mg, 830 mg, 835 mg, 840 mg, 845 mg, 850 mg, 855 mg, 860 mg, 865 mg, 870 mg, 875 mg, 880 mg, 885 mg, 890 mg, 895 mg, 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, 1000 mg, 1005 mg, 1010 mg, 1015 mg, 1020 mg, 1025 mg, 1030 mg, 1035 mg, 1040 mg, 1045 mg, 1050 mg, 1055 mg, 1060 mg, 1065 mg, 1070 mg, 1075 mg, 1080 mg, 1085 mg, 1090 mg, 1095 mg, 1100 mg, 1150 mg, 1200 mg, 1250 mg, 1300 mg, 1350 mg, or 1400 mg. In one embodiment, the anti-C5 agent (e.g., anti-C5 antibody, or antigen binding fragment thereof), is administered in a single dose. In another embodiment, multiple doses of the anti-C5 agent (e.g., anti-C5 antibody, or antigen binding fragment thereof), are administered.

The anti-C5 agent (e.g., anti-C5 antibody, or antigen binding fragment thereof), can be administered alone or in combination (e.g., separately or simultaneously) with one or more additional therapeutic agents. In one embodiment, the anti-C5 agent (e.g., anti-C5 antibody, or antigen binding fragment thereof), is administered in combination with no more than three additional agents. In another embodiment, the anti-C5 agent (e.g., anti-C5 antibody, or antigen binding fragment thereof), is administered in combination with no more than two additional agents. In another embodiment, the anti-C5 agent (e.g., anti-C5 antibody, or antigen binding fragment thereof), is administered in combination with no more than one additional agent. In another embodiment, the anti-C5 agent (e.g., anti-C5 antibody, or antigen binding fragment thereof), is administered alone.

### V. Methods Relating to Hepatic Ischemia Reperfusion Injury (IRI)

Ischemia reperfusion injury (IRI) is the phenomenon during which cellular damage in an organ, caused by hypoxia (oxygen deficiency in a tissue), is exacerbated after the restoration of oxygen delivery (see Papadopoulos, et al., Arch Trauma Res. 2013 August; 2(2): 63-70 and Elias-Miro M, et al., Ischemia-Reperfusion Injury Associated with Liver Transplantation in 2011: Past and Future. 2012). IRI is a dynamic process which involves the two interrelated phases of local ischemic insult and inflammation-mediated reperfusion injury (see Zhai Y, et al., Nat Rev Gastroenterol Hepatol. 2013; 10(2):79-89). This concept occurs in several organ systems such as the central nervous system, liver, heart, lung, intestine, skeletal muscle, and kidney (see Eltzschig H K, et al., Br Med Bull. 2004; 70:71-86). If severe enough, the inflammatory response after IRI can even result in systemic inflammatory response syndrome (SIRS) or multiple organ dysfunction syndrome (MODS) (see Videla L A, et al., World J Hepatol. 2009; 1(1):72-8).

Hepatic IRI is a frequent and major complication in clinical practice, which compromises liver function and increases postoperative morbidity, mortality, recovery, and overall outcome. Liver, being an organ with high energy requirements, is highly dependent on oxygen supply and susceptible to hypoxic or anoxic conditions (see Teoh N C, J Gastroenterol Hepatol. 2011; 26 Suppl 1:180-7). Hepatic IRI can be categorized into warm and cold ischemia. Warm ischemia occurs in the setting of transplantation, trauma, shock, and elective liver surgery, in which hepatic blood supply is temporarily interrupted. It may also occur in some types of toxic liver injury, sinusoidal obstruction and Budd-Chiari syndrome (see Fernandez V, et al., World J Hepatol. 2012; 4(4):119-28). Cold storage ischemia occurs during organ preservation before transplantation. Numerous factors contribute to hepatic IRI, including Kupffer cells (KC) activation, oxidative stress and upregulation of proinflammatory cytokine signaling (see van Golen R F, et al., J Gastroenterol Hepatol. 2013; 28(3):394-400). This variety of mechanisms, contribute to various extents to the overall pathophysiology.

Hepatic IRI also exists in the context of trauma, for example, hepatic resection in the hepatic trauma setting, especially in severe injuries (see Banga N R, et al. Br J Surg. 2005; 92(5):528-38). Intraoperative cessation of hepatic blood supply, by a variety of clamping maneuvers, is sometimes necessary during resection and inevitably exposes the liver to warm IRI. Furthermore, the liver with its role as the biochemical factory of sorts for the organism, as well as its anatomic and physiologic position, is vulnerable to the ischemia which is frequently encountered in patients with trauma.

During an ischemic period, several functional changes occur at the cellular level that promote cell injury (see Papadopoulos, et al., Arch Trauma Res. 2013 August; 2(2): 63-70 and Casillas-Ramirez A, et al., Life Sci. 2006; 79(20):1881-94). In particular, a decrease in oxidative phosphorylation, results in Adenosine-5′-triphosphate (ATP) depletion and derangements in calcium homeostasis (see De Groot H, et al., Transplant Proc. 2007; 39(2):481-4). The lack of oxygen to hepatocytes during ischemia also causes mitochondrial deenergization, alterations of H+ and Na+ homeostasis, and finally swelling of the sinusoidal endothelial cells (SEC), and the KC (see Massip-Salcedo M, et al., Liver Int. 2007; 27(1):6-16). Activation of KC with production of reactive oxygen species (ROS), upregulation of the inducible nitric oxide synthase (iNOS) in hepatocytes, and upregulation of proinflammatory cytokines, chemokines, and adhesion molecules resulting in neutrophil-mediated injury, are all major contributing events to the inflammation-associated damage (see Bilzer M, et al., J Hepatol. 2000; 32(3):508-15).

In one aspect, a method of treating hepatic ischemia reperfusion injury (IRI) in a patient who has experienced hepatic trauma (e.g., due to any type of physical injury, including surgery), comprising administering to the patient an effective amount of an anti-C5 agent (e.g., anti-C5 antibody, or antigen binding fragment thereof). In one embodiment, the treatment results in decreased hepatocyte apoptosis compared to a pre-treatment baseline (e.g., as assessed by single-stranded-DNA staining and/or western blot for cleaved caspase-3). In one embodiment, the treatment results in a 1.5-fold, 2-fold, 2.5-fold, 3-fold, or 3.5-fold decrease in hepatocyte apoptosis compared to a pre-treatment baseline.

In another embodiment, the treatment results in a decrease in one or more pro-inflammatory cytokines (e.g., IL-1β, IL-6, and/or TNFα) and/or one or more chemokines (e.g., CXCL-1 and/or CXCL-2) compared to a pre-treatment baseline (e.g., a 25%, 30% 40%, 50%, 60%, 70%, 80% or greater decrease). In another embodiment, the treatment results in a decrease in one or more pro-inflammatory cytokines (e.g., IL-1β, IL-6, and/or TNFα) and/or one or more chemokines (e.g., CXCL-1 and/or CXCL-2) to within normal levels or to within 10%, 15%, or 20% above what is considered the normal level.

In another embodiment, the treatment results in a decrease in one or more parenchymal damage markers (e.g., AST, ALT and/or T-bil) compared to a pre-treatment baseline (e.g., a 25%, 30% 40%, 50%, 60%, 70%, 80% or greater decrease). In another embodiment, the treatment results in a decrease in one or more parenchymal damage markers (e.g., AST, ALT and/or T-bil) to within normal levels or to within 10%, 15%, or 20% above what is considered a normal level for the marker.

In another embodiment, the treatment results in decreased neutrophil infiltration compared to a pre-treatment baseline (e.g., a 25%, 30% 40%, 50%, 60%, 70%, 80% or greater decrease). In another embodiment, the treatment results in decreased neutrophil infiltration to within normal levels of neutrophils or to within 10%, 15%, or 20% above what is considered the normal level.

In another embodiment, the treatment results in decreased platelet aggregation compared to a pre-treatment baseline (e.g., a 25% m 30% 40%, 50%, 60%, 70%, 80% or greater decrease). In another embodiment, the treatment results in decreased platelet aggregation to within normal levels of platelet aggregation or to within 10%, 15%, or 20% above what is considered the normal level.

In another embodiment, the treatment results in an at least one score improvement, as assessed by the Suzuki Scoring System for the assessment of liver damage following hepatic IRI set forth in Table 1 (see, e.g., Matthias Behrends, et al., J Gastrointest Surg. 2010 March; 14(3): 528-535, Suzuki S, et al., Transplantation, 1993; 55(6): 1265-72, and Suzuki S, et al., Transplantation. 1991; 52:979-98). For example, the patient may have (1) a score of 4 prior to treatment and a score of 3 after treatment, (2) a score of 3 prior to treatment and a score of 2 after treatment, (3) a score of 2 prior to treatment and a score of 1 after treatment, or (4) a score of 1 prior to treatment and a score of 0 after treatment. In another embodiment, the treatment results in a at least 2,3, or 4 score improvement. For example, the patient may have (1) a score of 4 prior to treatment and a score of 2 after treatment, (2) a score of 3 prior to treatment and a score of 1 after treatment, (3) a score of 2 prior to treatment and a score of 0 after treatment, (4) a score of 4 prior to treatment and a score of 1 after treatment, (5) a score of 3 prior to treatment and a score of 0 after treatment, or (6) a score of 4 prior to treatment and a score of 0 after treatment.

TABLE 1
Suzuki Scoring System

| Score | Congestion | Vacuolization | Necrosis |
|---|---|---|---|
| 0 | None | None | None |
| 1 | Minimal | Minimal | Single cell necrosis |
| 2 | Mild | Mild | −30% |
| 3 | Moderate | Moderate | −60% |
| 4 | Severe | Severe | > |

Also provided are methods for decreasing levels of one or more pro-inflammatory cytokines (e.g., IL-1β, IL-6, and/or TNFα) and/or one or more chemokines (e.g., CXCL-1 and/or CXCL-2) in a patient who has experienced hepatic trauma, by administering to the patient an effective amount of an anti-C5 agent (e.g., anti-C5 antibody, or antigen binding fragment thereof), thereby decreasing levels of the one or more pro-inflammatory cytokines and/or one or more chemokines in the patient compared to pre-treatment baseline levels. In one embodiment, the method results in a decrease in one or more pro-inflammatory cytokines (e.g., IL-1β, IL-6, and/or TNFα) and/or one or more chemokines (e.g., CXCL-1 and/or CXCL-2) compared to a pre-treatment baseline (e.g., a 25%, 30% 40%, 50%, 60%, 70%, 80% or greater decrease). In another embodiment, the method results in a decrease in one or more pro-inflammatory cytokines (e.g., IL-1β, IL-6, and/or TNFα) and/or one or more chemokines (e.g., CXCL-1 and/or CXCL-2) to within normal levels or to within 10%, 15%, or 20% above what is considered the normal level.

Further provided are methods of decreasing neutrophil infiltration in a patient who has experienced hepatic trauma (e.g., due to any type of physical injury, including surgery), by administering to the patient an effective amount of an anti-C5 agent (e.g., anti-C5 antibody, or antigen binding fragment thereof), thereby decreasing neutrophil levels compared to pre-treatment baseline neutrophil levels. In one embodiment, the method results in decreased neutrophil infiltration compared to a pre-treatment baseline (e.g., a 25%, 30% 40%, 50%, 60%, 70%, 80% or greater decrease). In another embodiment, the treatment results in decreased neutrophil infiltration to within normal levels of neutrophils or to within 10%, 15%, or 20% above what is considered the normal level.

The assessments described herein, for example, a decrease in hepatocyte apoptosis, cytokines, chemokines, parenchymal damage markers, neutrophil infiltration, and/or platelet aggregation) can be determined at any time after administration of the anti-C5 agent (e.g., anti-C5 antibody, or antigen binding fragment thereof). In one embodiment, the decrease is assessed 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 48, or 72 hours post treatment.

### VI. Methods Relating to Acute Liver Failure

Acute liver failure (also known as fulminant hepatic failure) is a loss of liver function (e.g., loss of function of 80-90% of liver cells) that occurs rapidly (e.g., in days or weeks), usually in a person who has no pre-existing liver disease. Acute liver failure is defined as a syndrome of acute hepatitis with evidence of abnormal coagulation (e.g., an international normalized ratio >1.5) complicated by the development of mental alteration (encephalopathy) within 26 weeks of the onset of illness in a patient without a history of liver disease (see, e.g., Polson J, Lee W M; American Association for the Study of Liver Disease. Hepatology 2005; 41:1179-1197 and Sleisenger & Fordtran's gastrointestinal and liver disease pathophysiology, diagnosis, management (PDF) (9th ed.)).

The term acute liver failure has replaced older terms such as fulminant hepatic failure, hyperacute liver failure, and subacute liver failure, which were used for prognostic purposes. Patients with hyperacute liver failure (defined as development of encephalopathy within 7 days of onset of illness) generally have a good prognosis with medical management, whereas those with subacute liver failure (defined as development of encephalopathy within 5 to 26 weeks of onset of illness) have a poor prognosis without liver transplant (see, e.g., O'Grady J G, et al., Lancet 1993; 342:273-275 and Ostapowicz G, et al; US Acute Liver Failure Study Group. Results of a prospective study of acute liver failure at 17 tertiary care centers in the United States. Ann Intern Med 2002; 137:947-954).

Acute liver failure occurs when liver cells are damaged significantly and are no longer able to function. Potential causes include: an acetaminophen overdose, prescription medications (e.g., antibiotics, nonsteroidal anti-inflammatory drugs and anticonvulsants), herbal supplements, (e.g., kava, ephedra, skullcap and pennyroyal), viruses (e.g., hepatitis A, hepatitis B, hepatitis E, Epstein-Barr virus, cytomegalovirus and herpes simplex virus), toxins (e.g., Amanita phalloides and carbon tetrachloride), autoimmune disease, vascular diseases (e.g., Budd-Chiari syndrome), metabolic diseases (e.g., Wilson's disease and acute fatty liver of pregnancy), cancer, and septic shock. However, many cases of acute liver failure have no apparent cause.

There are nearly 2,000 cases of acute liver failure each year in the United States, and it accounts for 6% of all deaths due to liver disease (see Lee W M, et al., Acute liver failure: summary of a workshop. Hepatology 2008; 47:1401-1415). It is more common in women than in men, and more common in white people than in other races. The peak incidence is at a fairly young age (e.g., between 35 to 45 years) (see, e.g., Singh et al., Cleveland Clinic Journal of Medicine. 2016 June; 83(6):453-462 and Bernal et al., N Engl J Med 2013; 369:2525-2534). The diagnosis of acute liver failure is based on physical examination, laboratory findings, patient history, and past medical history to establish mental status changes, coagulopathy, rapidity of onset, and absence of known prior liver disease respectively. Acute liver failure can cause serious complications, including excessive bleeding and pressure in the brain. Symptoms of acute liver failure, include, but are not limited to hepatic encephalopathy, impaired protein synthesis (e.g., as measured by levels of serum albumin and the prothrombin time in the blood), jaundice, pain in the upper right abdomen, abdominal swelling, nausea, vomiting, malaise, disorientation or confusion, and/or sleepiness. Acute liver failure can often cause complications, including: cerebral edema, bleeding and bleeding disorders, infections, and/or kidney failure.

Accordingly, in one aspect, methods of treating a patient who has been determined to have acute liver failure are provided, comprising administering to the patient an effective amount of an anti-C5 agent (e.g., anti-C5 antibody, or antigen binding fragment thereof). In one embodiment, the treatment produces at least one therapeutic effect selected from the group consisting of a reduction or cessation in hepatic encephalopathy, impaired protein synthesis, jaundice, pain in the upper right abdomen, abdominal swelling, nausea, vomiting, malaise, disorientation, confusion, and/or sleepiness.

One way to assess liver function is via albumin levels. Albumin is the most abundant protein produced by the liver. The typical value for serum albumin in blood is 3.4 to 5.4 grams per deciliter. A serum albumin below 3.4 grams per deciliter is considered low. Serum albumin levels are low during liver failure. Because of its long half-life (2-3 weeks), albumin is most useful in the assessment of chronic liver failure. Accordingly, in one embodiment, the methods described herein result in a shift towards normal levels of serum albumin.

In one aspect, methods of increasing serum albumin in a patient who has been determined to have acute liver failure are provided, comprising administering to the patient an effective amount of an anti-C5 agent (e.g., anti-C5 antibody, or antigen binding fragment thereof), thereby increasing serum albumin in the patient compared to a pre-administration baseline serum albumin level. In one embodiment the patient's serum albumin is below 3.4 grams per deciliter prior to administration of the anti-C5 agent (e.g., anti-C5 antibody, or antigen binding fragment thereof). In another embodiment, the patient's serum albumin is between 3.4 grams to 5.4 grams per deciliter after administration of the anti-C5 agent (e.g., anti-C5 antibody, or antigen binding fragment thereof).

Another means for measuring liver function is Prothrombin Time (PT) and/INR (International Normalized Ratio). The liver produces the majority of coagulation proteins needed in blood clotting cascade. Severe liver injury leads to reduction of liver synthesis of clotting factors and consequently prolonged PT or an increased INR, which is a method to homogenize PT level reporting across the world. Because clotting factors have shorter half-life than albumin, PT (or INR) is useful in assessing the presence of both acute and chronic liver failure.

A Prothrombin time test (also referred to as a “PT” or “pro time” test) is a blood test that measures how long it takes blood to clot. A prothrombin time test can be used to check for bleeding problems, as well as to check whether medicine to prevent blood clots is working. Blood clotting factors are needed for blood to coagulate (clot). Prothrombin, or factor II, is one of the clotting factors made by the liver. Vitamin K is needed to make prothrombin and other clotting factors. Prothrombin time is an important test because it checks to see if five different blood clotting factors (factors I, II, V, VII, and X) are present. The prothrombin time is made longer by: blood-thinning medicine (e.g., warfarin), low levels of blood clotting factors, a change in the activity of any of the clotting factors, the absence of any of the clotting factors, inhibitors, and/or an increase in the use of the clotting factors. An abnormal prothrombin time is often caused by liver disease or injury or by treatment with blood thinners.

The prothrombin time is a measure of the integrity of the extrinsic and final common pathways of the coagulation cascade. This consists of tissue factor and factors VII, II (prothrombin), V, X, and fibrinogen. The test is performed by adding calcium and thromboplastin, an activator of the extrinsic pathway, to the blood sample then measuring the time (in seconds) required for fibrin clot formation. The normal reference range for prothrombin time is 9.5-13.5 seconds. However, the normal range is highly variable and dependent on the laboratory performing the test. Accordingly, in one embodiment, the methods described herein result in the patient having a Prothrombin Time (PT) between 9.5 to 13.5 seconds.

In another aspect, methods of decreasing PT in a patient who has been determined to have acute liver failure are provided, comprising administering to the patient an effective amount of an anti-C5 agent (e.g., anti-C5 antibody, or antigen binding fragment thereof), thereby decreasing PT time in the patient compared. In one embodiment, the patient's PT is >13.5 seconds prior to administration of the anti-C5 agent (e.g., anti-C5 antibody, or antigen binding fragment thereof). In another embodiment, the patient's PT is between is 9.5 to 13.5 seconds after administration of the anti-C5 agent (e.g., anti-C5 antibody, or antigen binding fragment thereof).

The prothrombin time can have significant inter-laboratory variability influenced by the instrument, and more importantly, the reagent used. In an effort to offset variation in thromboplastin reagent, and enhance standardization of PT in patients receiving warfarin, the World Health Organization (WHO) introduced the International normalized ratio (INR) in 1983 (see Tajiri K, et al., J Cardiol. 2015; 65(3):191-6, Ohara M, et al., PLoS One. 2014; 9(8):e105891, Rohrer M J, et al., Crit Care Med. 1992; 20(10):1402-5, and Levy J H, et al., Clin Lab Med. 2014; 34(3):453-77). The INR is intended to standardize PT, such that a PT generated from one laboratory would yield an INR value comparable to that generated from any other laboratory in the world (see Ng V L, Clin Lab Med. 2009; 29(2):253-63). It is basically a mathematical conversion of a patient's PT that accounts for the sensitivity of the reagent used in a given laboratory by factoring in the International Sensitivity Index (ISI) of assigned by its manufacturer (see Kamal A H, et al., Mayo Clin Proc. 2007; 82(7):864-73). The ISI is a measure of a reagent's sensitivity to a reduction in Vitamin K-dependent factors (II, VII, IX, X) compared with the WHO International Reference Preparation. The INR is then calculated using the following formula: INR=[Patient PT/Mean PT]ISI. In this formula, patient PT is measured prothrombin time, mean PT is geometric mean PT of at least 20 healthy subjects of both sexes tested at a particular laboratory, and ISI is International Sensitivity Index that is specific to each reagent-instrument combination. A normal INR is 0.8 to 1.1. Each increase of 0.1 means the blood is slightly thinner (e.g., it takes longer to clot). Acute liver failure is defined as a syndrome of acute hepatitis with evidence of abnormal coagulation (e.g., an international normalized ratio >1.5). Accordingly, in one embodiment, the methods described herein result in the patient having an International Normalized Ratio (INR) between 0.8 to 1.1. In another embodiment, the treatment results in the patient having a PT between 9.5 to 13.5 seconds and an INR between 0.8 to 1.1.

In a further aspect, methods of decreasing INR in a patient who has been determined to have acute liver failure are provided, the method comprising administering to the patient an effective amount of an anti-C5 agent (e.g., anti-C5 antibody, or antigen binding fragment thereof), thereby decreasing INR in the patient. In one embodiment, the patient's INR is >1.5 prior to administration of the anti-C5 agent (e.g., anti-C5 antibody, or antigen binding fragment thereof). In another embodiment, the patient's INR is between 0.8 to 1.1 after administration of the anti-C5 agent (e.g., anti-C5 antibody, or antigen binding fragment thereof).

The assessments described herein, for example, a decrease in PT, INR, and/or physical symptoms, and/or an increase in serum albumin levels, can be determined at any time after administration of the anti-C5 agent (e.g., anti-C5 antibody, or antigen binding fragment thereof). In one embodiment, the decrease or increase is assessed 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 48, or 72 hours post treatment.

Different criteria have been used to identify patients with poor prognosis who may eventually need to undergo liver transplant (see Singh et al., Cleveland Clinic Journal of Medicine. 2016 June; 83(6):453-462). Accordingly, in one embodiment, the methods described herein result in a change from baseline, as assessed via The King's College criteria system, the Model for End-Stage Liver Disease (MELD) scoring system, the Acute Physiology and Chronic Health Evaluation (APACHE) II scoring system, and/or the Clichy criteria. Each of these criteria are disclosed below in further detail.

The King's College criteria system is the most commonly used for prognosis (see Clemmesen J O, et al., Hepatology 1999; 29:648-653, Pauwels A, et al., J Hepatol 1993; 17:124-127, Anand A C, et al., J Hepatol 1997; 26:62-68, Schmidt L E, et al., Hepatology 2007; 45:789-796, and Bernuau J, et al., Hepatology 1986; 6:648-651). Its main drawback is that it is applicable only in patients with encephalopathy, and when patients reach this stage, their condition often deteriorates rapidly, and they die while awaiting liver transplant.

In their seminal paper, O'Grady et al. explored data from 588 patients with ALF treated between 1983 and 1985 at the Liver Unit of the King's College in London, UK (see O'Grady J G, et al., Gastroenterology 1989; 97:439-445). Multivariate analysis of this large cohort of patients revealed that separate predictors for transplant-free survival apply to (a) paracetamol-induced and (b) non-paracetamol induced ALF (Table 2) (see Renner, Forum on Liver Transplantation, Journal of Hepatology 46 (2007) 553-582).

TABLE 2
King's College Criteria for Selection of ALF patients for Liver Transplantation

| | |
|---|---|
| Paracetamol-induced ALF | Arterial blood pH <7.30 (irrespective of grade of encephalopathy) |
| | OR all of the following: Prothrombin time >100 s (INR >6.5), |
| | serum creatinine >300 μmol/L, and Grade III or IV hepatic |
| | encephalopathy |
| Non-Paracetamol induced | Prothrombin time >100 s (INR > 6.5) (irrespective of grade of |
| ALF | encephalopathy) OR any 3 of the following (irrespective of grade of |
| | encephalopathy): Age <10 or >40 years, Etiology: non-A/non-B |
| | hepatitis, drug-induced, Duration of jaundice to encephalopathy >7 |
| | days, Prothrombin time >50 (INR > 3.5), Serum bilirubin >300 μmol/L |

The model was validated in an independent cohort of 175 ALF patients treated between 1986 and 1987 at the same institution. Positive predictive values (i.e., the observed mortality rate in those patients predicted to die) were 84% and 98%, and negative predictive values (i.e., the observed survival rate in those patients predicted to survive) were 86% and 82% for paracetamol-induced and non-paracetamol induced ALF, respectively. This translates into predictive accuracies of 85% and 95%. The King's college criteria are based on simple parameters readily available at admission and are widely used for selecting ALF patients for liver transplantation worldwide. They are however derived from a cohort of patients treated now more than 30 years ago, and critical care management of ALF patients has made dramatic progress over the past two decades.

The Model for End-Stage Liver Disease (MELD) score is an alternative to the King's College criteria. A high MELD score on admission signifies advanced disease, and patients with a high MELD score tend to have a worse prognosis than those with a low score (see Schmidt L E, et al., Hepatology 2007). The MELD scoring system was initially developed to predict mortality within three months of surgery in patients who had undergone a transjugular intrahepatic portosystemic shunt (TIPS) procedure and was subsequently found to be useful in determining prognosis and prioritizing for receipt of a liver transplant (see Malinchoc, et al., (2000) Hepatology. 31 (4): 864-71, Kamath, P. et al., (2001) Hepatology. 33 (2): 464-70, and Kamath, et al., (2007), Hepatology. 45 (3): 797-805). This score is used by the United Network for Organ Sharing (UNOS) and Eurotransplant for prioritizing allocation of liver transplants (see Kamath, et al., (2007), Hepatology. 45 (3): 797-805 and Jung, G. E et al., (2008), Der Chirurg. 79 (2): 157-63).

MELD uses the patient's values for serum bilirubin, serum creatinine, and the international normalized ratio for prothrombin time (INR) to predict survival. It is calculated according to the following formula: MELD=3.78×ln[serum bilirubin (mg/dL)]+11.2×ln[INR]+9.57×ln[serum creatinine (mg/dL)]+6.43 (see Kamath, et al., (2007), Hepatology. 45 (3): 797-805). MELD scores are reported as whole numbers, so the result of the equation above is rounded. UNOS has made the following modifications to the score (see UNOS (2009 Jan. 28). “MELD/PELD calculator documentation”): If the patient has been dialyzed twice within the last 7 days, then the value for serum creatinine used should be 4.0 mg/dL. Any value less than one is given a value of 1 (i.e., if bilirubin is 0.8 a value of 1.0 is used) to prevent subtraction from any of the three factors, since the natural logarithm of a positive number below 1 (greater than 0 and less than 1) yields a negative value. The etiology of liver disease was subsequently removed from the model because it posed difficulties, such as how to categorize patients with multiple causes of liver disease. Modification of the MELD score by excluding etiology of liver disease did not significantly affect the model's accuracy in predicting three-month survival.

In interpreting the MELD Score in hospitalized patients, the 3 month observed mortality (considering 3437 adult liver transplant candidates with chronic liver disease who were added to the OPTN waiting list at 2A or 2B status between November, 1999, and December, 2001) is described by Wiesner, et al. (United Network for Organ Sharing Liver Disease Severity Score Committee (2003). “Model for end-stage liver disease (MELD) and allocation of donor livers”. Gastroenterology. 124 (1): 91-6) and set forth below in Table 3. Patients with MELD scores greater than 24 who are reasonable liver transplant candidates are probably best served by foregoing transjugular intrahepatic portosystemic shunt (TIPS) placement.

TABLE 3
MELD Scoring Criteria

| Score | Observed Mortality |
|---|---|
| 40 or more | 71.3% observed mortality |
| 30-39 | 52.6% observed mortality |
| 20-29 | 19.6% observed mortality |
| 10-19 | 6.0% observed mortality |
| <9 | <1.9% observed mortality |

The Acute Physiology and Chronic Health Evaluation (APACHE) scores can also be used and are considered more sensitive than the King's College criteria (see Larson A M, Polson J, Fontana R J, et al.; Hepatology 2005; 42:1364-1372). APACHE II and III scores were developed by Knaus et al in 1985 and 1991, respectively, and are being used mainly for critically ill patients of all disease categories admitted to the intensive care units (ICUs) (see Knaus W A, et al., Crit Care Med. 1985; 13:818-829 and Knaus W A, et al., Chest. 1991; 100:1619-1636). The two scoring systems differ in how chronic health status is assessed, in the number of physiologic variables included (12 versus 17), and in the total score. Specific parameters of liver function (i.e., serum bilirubin and albumin) are included only in the APACHE III scoring system. Some prognostic variables (e.g., prothrombin time) and other indicators of responses to therapy (e.g., blood units transfused) which are known to be important outcome predictors in cirrhotic patients are not measured by the acute physiology scores (see Infante-Rivard C, et al., Hepatolology. 1987; 7:660-664, Ferro D, et al., Scand. J. Gastroenterol. 1992; 27:852-856, LeMoine O, et al., Gut. 1992; 33:1381-1385, and Christensen E, et al., Scand J Gastroenterol. 1989; 24:999-1006). APACHE II and III scores have been successfully used to risk stratify cirrhotic patients admitted to medical ICUs (see Zauner C A, et al., Intensive Care Med. 1996; 22:559-563, Zauner C, et al., Eur J Gastroenterol Hepatol. 2000; 12:517-522, Zimmerman J E, et al., Hepatology. 1996; 23:1393-1401, Wehler M, et al, Hepatology. 2001; 34:255-261, and Aggarwal A, et al., Chest. 2001; 119:1489-97).

To calculate the APACHE II score, twelve common physiological and laboratory values (temperature, mean arterial pressure, heart rate, respiratory rate, oxygenation (PaO₂ or A-aDo₂), arterial pH, serum sodium, serum potassium, serum creatinine, haematocrit, white blood cell count and Glasgow coma score) are marked from 0 to 4, with 0 being the normal, and 4 being the most abnormal (see Knaus W A, et al., Crit Care Med. 1985; 13:818-829). The sum of these values is added to a mark adjusting for patient age and a mark adjusting for chronic health problems (severe organ insufficiency or immunocompromised patients) to arrive at the APACHE II score.

APACHE III scores range from 0 to 299 and are derived from marks for the extent of abnormality of 17 physiologic measurements (the acute physiology score), adjusts for age, and adjusts for seven comorbidities that reduce immune function and influence hospital survival (see Knaus W A, et al., Chest. 1991; 100:1619-1636). The 17 physiological variables include eleven laboratory parameters (haematocrit, white blood cell count, serum creatinine, serum BUN, serum sodium, serum albumin, serum bilirubin, blood glucose, PaO₂, A-aDO₂, and a scoring for acid-base abnormalities), five vital signs (pulse, mean blood pressure, temperature, respiratory rate, urine output) and a modified Glasgow coma score.

The Clichy criteria can also be used (see Pauwels A, et al., J Hepatol 1993; 17:124-127 and Bernuau J, et al., Hepatology 1986; 6:648-651). Bernuau et al. reported in 1986 on 115 patients with HBV associated acute liver failure, mostly treated during the 1970s, and showed by multivariate analysis that factor V level, patient's age, absence of HBsAg in serum and serum α-fetoprotein concentration were independent predictors of survival (see Bernuau J, et al., Hepatology 1986; 6:648-651). These parameters were adopted by Bismuth et al. for selection of patients for orthotopic liver transplantation (OLT) in patients admitted for acute liver failure to the liver unit at Paul Brousse hospital in Paris between 1986 and 1991 (see Bismuth H, et al., The Paul Brousse experience. Ann Surg 1995; 222:109-119). The so called Clichy criteria are the presence of hepatic encephalopathy AND Factor V level of <20% (if patient's age <30 years) OR <30% (if patient's age ≥30 years). Of 139 patients with acute liver failure who met the criteria, 1 recovered, 22 died awaiting transplantation and 116 were transplanted with a 1-year survival of 81% in those receiving an ABO compatible whole liver graft without steatosis (see Renner, Forum on Liver Transplantation, Journal of Hepatology 46 (2007) 553-582)). This data seems to indicate that the Clichy criteria are able to select quite accurately the acute liver failure patients requiring a liver transplant. They are widely used in France for that purpose. The study does not, however, allow drawing conclusions as to the mortality in those who did not fulfill the criteria.

In addition to helping establish the cause of acute liver failure, liver biopsy can also be used as a prognostic tool. Hepatocellular necrosis greater than 70% on the biopsy predicts death with a specificity of 90% and a sensitivity of 56% (see Donaldson B W, et al. Hepatology 1993; 18:1370-1376). Hypophosphatemia has been reported to indicate recovering liver function in patients with acute liver failure (see Schmidt L E, et al., Hepatology 2002; 36:659-665). As the liver regenerates, its energy requirement increases. To supply the energy, adenosine triphosphate production increases, and phosphorus shifts from the extracellular to the intracellular compartment to meet the need for extra phosphorus during this process. A serum phosphorus level of 2.9 mg/dL or higher appears to indicate a poor prognosis in patients with acute liver failure, as it signifies that adequate hepatocyte regeneration is not occurring.

### VII. Kits

Also provided herein are kits which include a pharmaceutical composition containing an anti-C5 agent (e.g., anti-C5 antibody, or antigen binding fragment thereof, such as eculizumab or ravulizumab), and a pharmaceutically-acceptable carrier, in a therapeutically effective amount adapted for use in the methods described herein. The kits optionally also can include instructions, e.g., comprising administration schedules, to allow a practitioner (e.g., a physician, nurse, or patient) to administer the composition contained therein to a patient who has experienced hepatic trauma (e.g., due to any type of physical injury, including surgery) or to a patient determined to have acute liver failure.

In one embodiment, a kit for treating or preventing hepatic ischemia reperfusion injury (RI) in a patient is provided, the kit comprising: (a) a dose of an anti-C5 agent (e.g., anti-C5 antibody, or antigen binding fragment thereof); and (b) instructions for using the anti-C5 agent (e.g., anti-C5 antibody, or antigen binding fragment thereof), in any of the methods described herein. In another embodiment, a kit for treating a patient who has been determined to have acute liver failure is provided, the kit comprising: (a) a dose of an anti-C5 agent (e.g., anti-C5 antibody, or antigen binding fragment thereof); and (b) instructions for using the anti-C5 agent (e.g., anti-C5 antibody, or antigen binding fragment thereof), in any of the methods described herein.

The following examples are merely illustrative and should not be construed as limiting the scope of this disclosure in any way as many variations and equivalents will become apparent to those skilled in the art upon reading the present disclosure.

The contents of all references, Genbank entries, patents and published patent applications cited throughout this application are expressly incorporated herein by reference.

### Examples

### Example 1: Pre-Clinical Studies in Mice

Pre-clinical murine studies were conducted to assess the effects of C5 blockade using BB5.1 mAb, a mouse IgG1 isotype antibody that specifically binds to C5 in mice and inhibits both C5a and C5b-9 activity (see Wang Y, et al., Proc. Natl. Acad. Sci. USA 1996 August; 93: 8563-8). BB5.1 mAb has an inhibitory effect of terminal complement activity comparable to eculizumab, with cross-reactivity to C5 in rats (see Thomas T C, et al., Molecular Immunology 1996; 33(17/18): 1389-401).

The primary objective was to verify the therapeutic effect of the anti-complement C5 antibody against critical liver diseases, including both warm and cold ischemia/reperfusion injury of the liver in liver transplantation, as well as in liver surgeries. The secondary objective was to verify the therapeutic effect of the anti-complement C5 antibody against critical liver diseases, such as acute liver failure/fulminant hepatitis.

### 1. Mouse Model of Warm Ischemia-Reperfusion Injury:

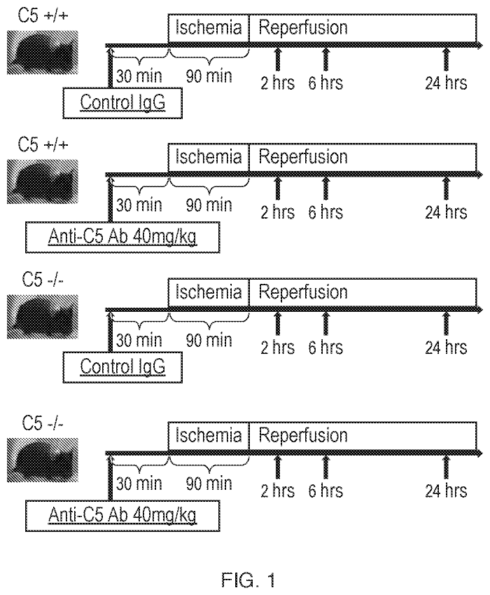

C5-knockout (KO, B10D2/oSn) and the corresponding wild-type (B10D2/nSn) mice were exposed to 70% partial hepatic ischemia for 90 minutes to the left and median lobes. All mice were anesthetized with isoflurane via a small animal anesthetizer (MK-A110, Muromachi Kikai Co., Ltd., Tokyo, Japan). The body temperature was maintained at 36.5±0.5° C. with a heating pad. After laparotomy with a midline incision, the left and median lobes were mobilized, and the vascular pedicle into those lobes was carefully encircled and clamped for 90 minutes using an atraumatic microvascular clip. Reperfusion was initiated by removing the clip. Prior to ischemia, either anti-C5 antibody BB5.1 mAb (20, 40, and 60 mg/kg) or control immunoglobulin G (IgG) was intravenously administered 30 minutes before ischemia, as shown in FIG. 1. Control WT/KO mice was pretreated with control immunoglobulin G (IgG). Sham-operated mice underwent the same procedure but without vascular occlusion. A C5a-receptor antagonist (C5aR-Ant: PMX53) was administered to WT mice in certain instances to clarify the dominant cascade (C5a or C5b) in hepatic ischemia/reperfusion injury (RI).

After 2, 6, and 24 hours of reperfusion, the mice are sacrificed and plasma Clq, Ba, C5a and C5b-9 Terminal Complement Complex C5b-9 were evaluated by ELISA. The hemolytic activity of complement was estimated from the degree of hemolysis of unsensitized sheep erythrocytes after incubation of mouse serum in the presence of zymosan. The mechanism of hemolysis is so-called the reactive lysis (deviated lysis, bystander lysis), in which erythrocytes are lysed when serum complement activation proceeds not on the erythrocyte membrane, but in the fluid phase close to the erythrocytes. In addition, markers of parenchymal damage (i.e., AST, ALT, and T-Bil) and biochemical markers of Microangiopathy (i.e., platelet count, LDH release, plasma ADAMTS13 activity, and Unusually-Large von Willebrand factor (UL-vWF) multimer) were assessed. Serum alanine aminotransferase (sALT) levels in peripheral blood (an indicator of hepatocellular injury), were measured by a standard spectrophotometric method with an automated clinical analyzer. Hepatic microcirculation measured by Laser Doppler Flowmetry, 02C0 (oxygen to see), as described in www.lea.de/eng/indexe.html. Cytokines and chemokines (i.e., TNF-α, IL-1β, IL-6, IL-10, and CXCL-2) were also assessed.

The following histological assessments were conducted: polymorphonuclear leukocyte (PMN) infiltration, Liver Damage quantified by Suzuki's Score, TUNEL assay, and C4d immunostaining. Liver paraffin sections (4-mm thick) were stained with hematoxylin-eosin (H & E). The severity of liver IRI (e.g., necrosis, sinusoidal congestion, and centrilobular ballooning) was blindly graded with a modified Suzuki's criteria on a scale from 0 to 4.

After deparaffinization of liver sections, the antigen was retrieved with citrate buffer (10 mM, pH 6.0). After blocking with Protein Block Serum-Free (X0909, DAKO, Tokyo, Japan) for 30 minutes, the sections were incubated with rat monoclonal antibodies (mAbs) against mouse Ly6-G, ssDNA, CD11b, and F4/80. After incubation with biotinylated rabbit anti-rat IgG, immunoperoxidase (VECTASTAIN Elite ABC Kit, Vector Labs, Burlingame, Calif.) was applied to the sections. Positive cells were counted blindly at 10 high-power field (HPF)/section (×400). Negative controls were prepared by incubation with normal rat IgG instead of the first antibody.

To evaluate platelet aggregation in hepatic parenchyma, rat mAb against mouse CD41 was applied on liver frozen sections. After incubation with Alexa 488-conjugated goat anti-rat IgG, the stained sections were covered with Vectashield mounting medium containing 4,6-diamidino-2-phenylindole (Vector Laboratories, Burlingame, Calif., USA). The sections were observed with a BZ-9000 fluorescence microscope (Keyence, Osaka, Japan). Positive cells were counted blindly at 10 high-power field (HPF)/section (×400). The CD41-positive area was quantified using Image J software (National Institutes of Health, Bethesda, Md., USA).

Total RNA was extracted from the liver tissue using the RNeasy Kit (Qiagen, Venlo, the Netherlands) and complementary DNA was prepared by Omniscript RT kit (Qiagen). Quantitative RT-PCR was performed using the StepOnePlus Real-Time PCR System (Life Technologies, Tokyo, Japan). Target gene expression was calculated by the ratio to the housekeeping gene, glyceraldehyde 3-phosphate dehydrogenase (GAPDH).

All data are expressed as means±standard error of mean (SEM). Differences among experimental groups were analyzed using one-way analysis of variance (ANOVA) for unpaired data, followed by post-hoc tests, if appropriate, to compare the treated animals with WT-control group. P<0.05 was considered statistically significant.

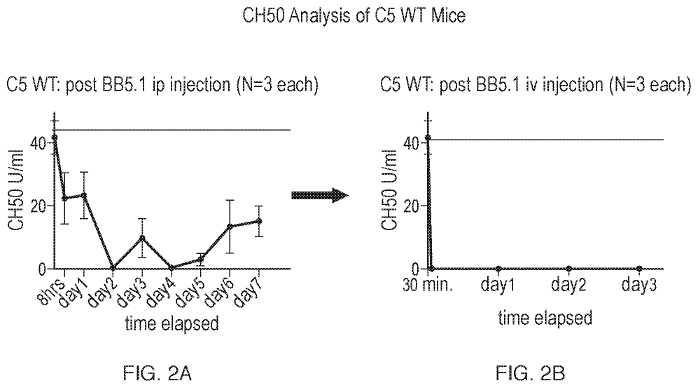

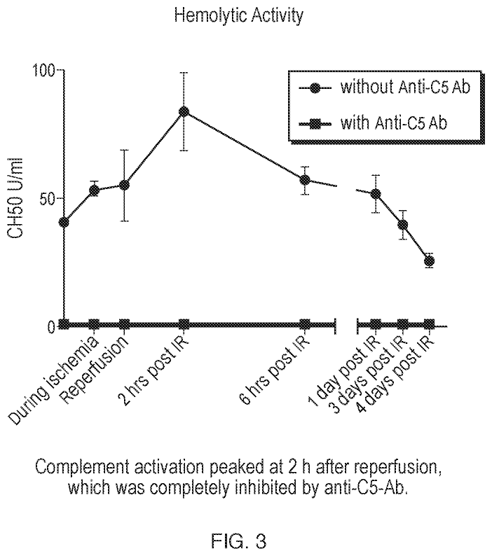

FIGS. 2A-2B depict the effect of BB5.1 on CH50 administered i.p. (FIG. 2A) or i.v. (FIG. 2B) to C5 WT mice. A single intravenous injection of BB5.1 completely suppressed CH50 for at least three days, whereas the effect by i.p. injection was not sufficient. FIG. 3 depicts hemolytic activity (CH50 U/ml) during ischemia, during reperfusion, 2 hours post IRI, 6 hours post IRI, 1 day post IRI, 3 days post IRI, and 4 days post IRI, with and without administration of an anti-C5 antibody. As shown in FIG. 3, complement activation peaked at 2 hours after reperfusion, which was completely inhibited by administration of the anti-C5 mAb.

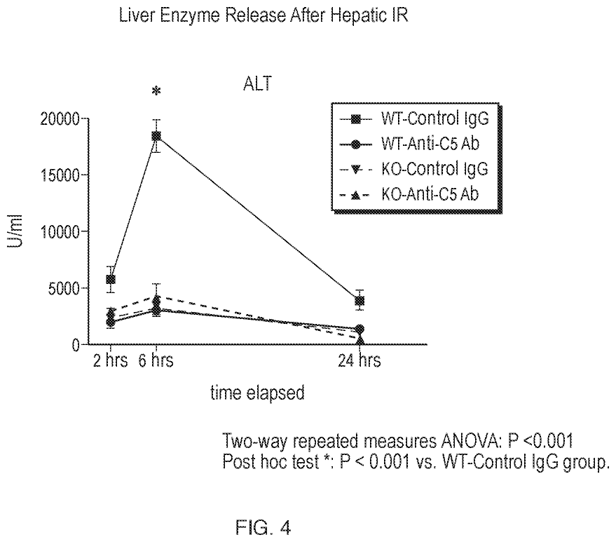

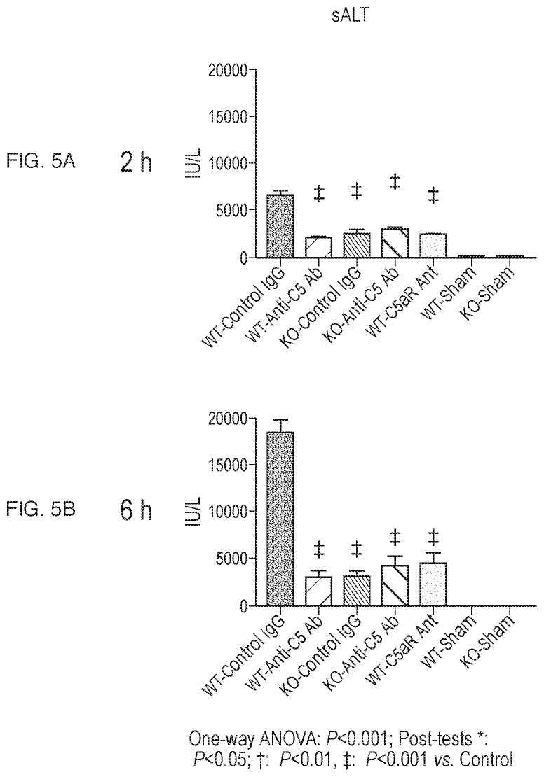

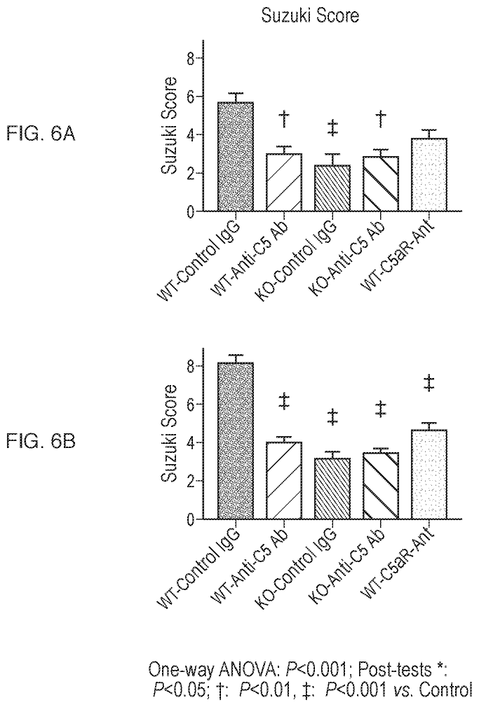

FIG. 4 depicts ALT (U/ml) release after hepatic ischemia-reperfusion for up to 24 hours for WT-control IgG, WT-Anti-C5 Ab, KO-Control IgG, and KO-Anti-C5 Ab. FIGS. 5A-5B depict ALT release (U/ml) at 2 hours (FIG. 5A) and 6 hours (FIG. 5B) after hepatic ischemia-reperfusion for WT-control IgG, WT-Anti-C5 Ab, KO-Control IgG, KO-Anti-C5 Ab, WT-C5aR-Ant, WT-Sham, and KO-Sham.

FIG. 6A-6B depict the histopathological evaluation as assessed by Suzuki Score for WT-control IgG, WT-Anti-C5 Ab, KO-Control IgG, KO-Anti-C5 Ab, and WT-C5aR-Ant at 2 hours (FIG. 6A) and 6 hours (FIG. 6B) post ischemia-reperfusion.

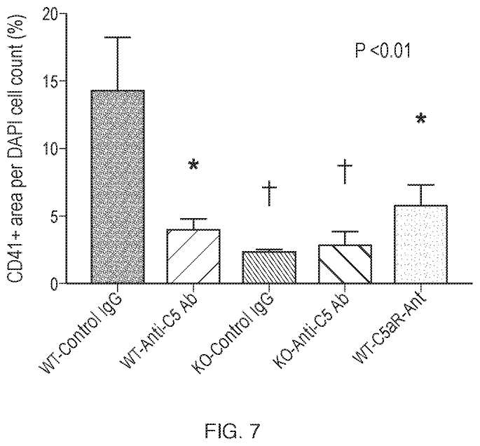

FIG. 7 depicts CD41 staining for WT-control IgG, WT-Anti-C5 Ab, KO-Control IgG, KO-Anti-C5 Ab, and WT-C5aR-Ant2 hours post IRI. As shown in FIG. 7, platelet aggregation in hepatic sinusoids was significantly lowered by Anti-C5 Ab, C5aR-antagonist, and in KO-Anti-C5 Ab.

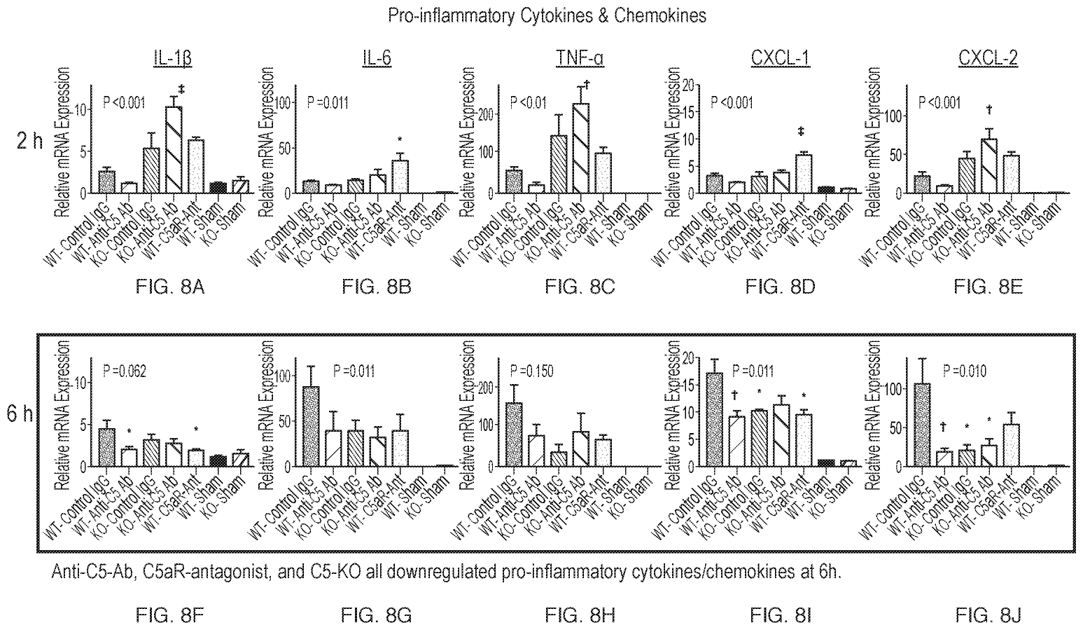

FIGS. 8A-8J depict IL-10 (FIG. 8A and FIG. 8F), IL-6 (FIG. 8B and FIG. 8G), TNF-α (FIG. 8C and FIG. 8H), CXCL-1 (FIG. 8D and FIG. 8I), and CXCL-2 (FIG. 8E and FIG. 8J, as assessed by qRT-PCR, at 2 hour and 6 hours post ischemia-reperfusion. As shown in FIGS. 8A-8J, Anti-C5 Ab, C5aR-antagonist, and KO-Anti-C5 Ab all downregulated pro-inflammatory cytokines/chemokines at 6 hours.

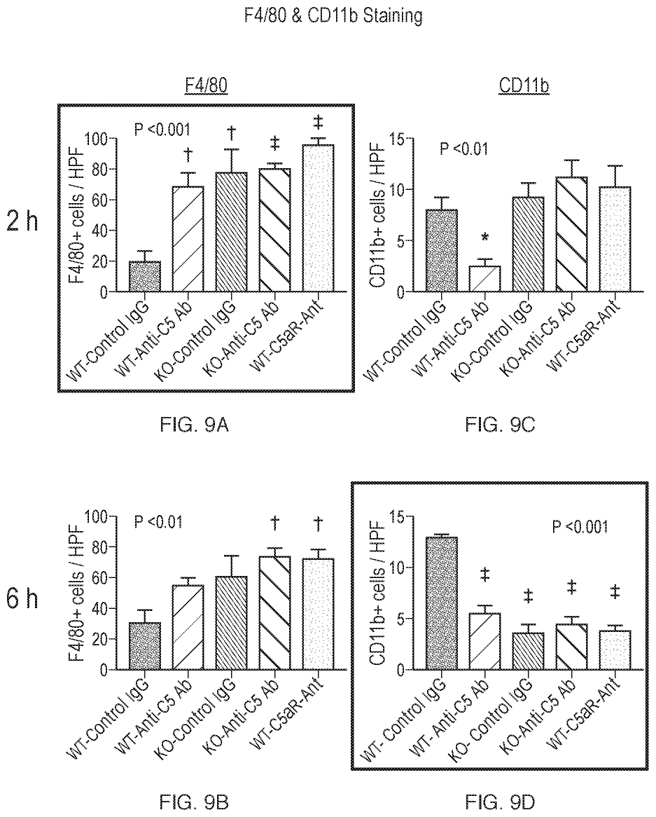

FIGS. 9A-9D depict F4/80+ cells (whole liver macrophage) (FIG. 9A and FIG. 9B) and CD11b cells (infiltrating macrophages) (FIG. 9C and FIG. 9D) for WT-control IgG, WT-Anti-C5 Ab, KO-Control IgG, KO-Anti-C5 Ab, and WT-C5a-Ant at 2 hours and 6 hours post ischemia-reperfusion. F4/80+ cells decreased in IRI and CD11b cells increased in IRI. Anti-C5 Ab, C5aR-antagonist, and C5 knockout all preserved F4/80+ cells at 2 hours and suppressed activation of CD11b+ cells at 6 hours.

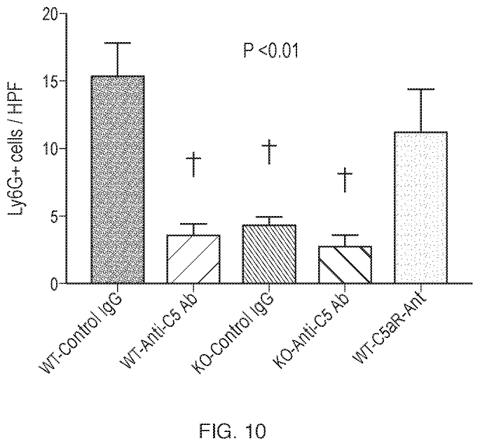

FIG. 10 depicts Ly6G cell staining for WT-control IgG, WT-Anti-C5 Ab, KO-Control IgG, KO-Anti-C5 Ab, and WT-C5a-Ant at 6 hours post ischemia-reperfusion. Anti-C5 Ab and the WT-C5a-Ant reduced neutrophil infiltration. There was a greater improvement with total C5-inhibition than C5aR antagonism alone.

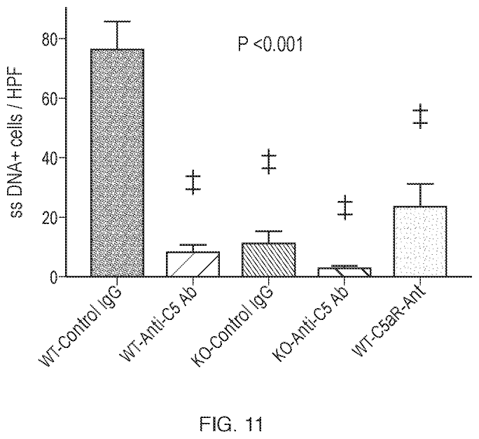

FIG. 11 depicts ssDNA staining for WT-control IgG, WT-Anti-C5 Ab, KO-Control IgG, KO-Anti-C5 Ab, and WT-C5a-Ant at 6 hours post ischemia-reperfusion. Again, Anti-C5 Ab and the WT-C5a-Ant reduced neutrophil infiltration. There was a greater improvement with total C5-inhibition than C5aR antagonism alone.

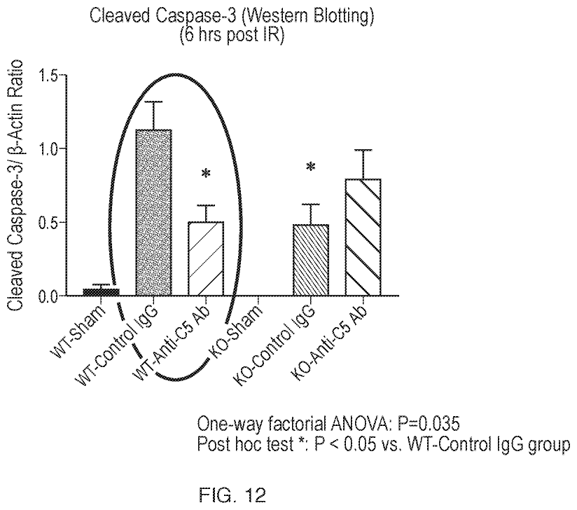

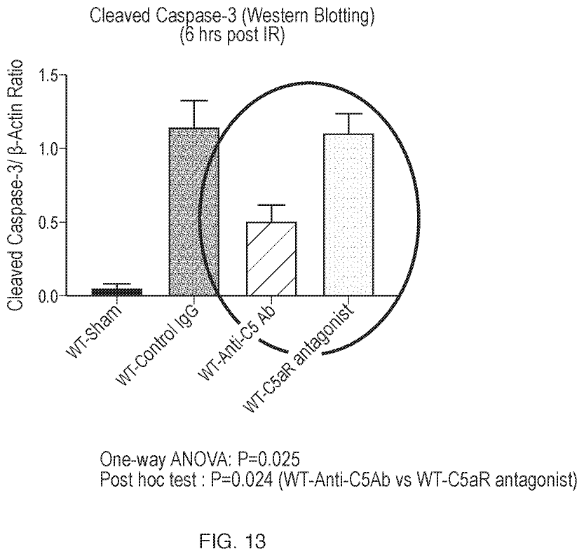

FIG. 12 depicts cleaved caspase-3 for WT Sham, WT-control IgG, WT-Anti-C5 Ab, KO Sham, KO-Control IgG, and KO-Anti-C5 Ab at 6 hours post ischemia-reperfusion, as assessed by Western Blotting. FIG. 13 depicts cleaved caspase-3 for WT Sham, WT-control IgG, WT-Anti-C5 Ab, and WT-C5aR-Ant at 6 hours post ischemia-reperfusion, as assessed by Western Blotting.

Hemolytic assays revealed that complement activation was completely inhibited by an intravenous administration of anti-C5-Ab (40 mg/kg) for at least 3 days. Serum ALT was significantly lowered in [WT+Anti-C5-Ab] and KO animals ([KO+Control-IgG] and [KO+Anti-C5-Ab]) than that in the control [WT+Control-IgG] at 2 and 6 hours after reperfusion (all P<0.001). Histopathological analysis also showed significantly less tissue damage by C5-knockout and anti-C5-Ab (P<0.001) than in the control. Immunohistochemistry for CD41 demonstrated that platelet aggregation in hepatic sinusoids was significantly less by anti-C5-Ab at 2 hours after reperfusion (P<0.01). Moreover, C5-inhibition significantly down-regulated pro-inflammatory cytokines (IL-1, IL-6, and TNF-α and chemokines (CXCL-1 and -2), followed by significantly less neutrophil infiltration at 6 hours (P<0.001). Oxidative damage marker, 8-hydroxy-2-deoxyguanosine, was also significantly decreased by C5-inhibition (P<0.01). Single-stranded-DNA staining and western-blot for cleaved caspase-3 both demonstrated anti-C5-Ab significantly decreased hepatocyte apoptosis (P<0.01 and <0.05, respectively). C5a-receptor antagonist exerted comparable protection with anti-C5-Ab in most parameters, however, neutrophil infiltration and hepatocyte apoptosis were significantly-more improved by total C5-inhibition than by C5a-receptor antagonism only.

Anti-C5 antibody significantly attenuated hepatic IRI, predominantly via C5a-mediated cascade, not only by inhibiting platelet aggregation and cytokines/chemokines production during early phase, but also by attenuating subsequent neutrophil infiltration, oxidative damage, and hepatocyte apoptosis during the late phase of reperfusion.

### 2. Mouse Model of Acute/Fulminant Liver Failure

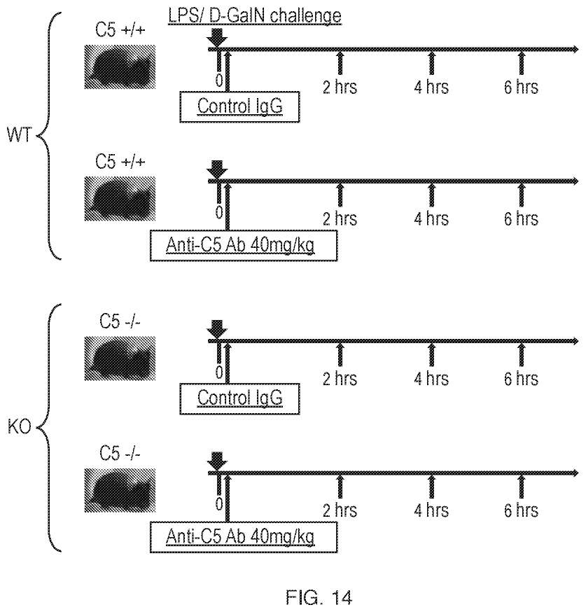

Male C5 deficient (KO, B10D2/oSn) and the corresponding wild-type (B10D2/nSn) mice (9-12 weeks-old) were subjected to LPS/D-GaIN challenge to induce acute fulminant Liver Failure (ALF), as shown in FIG. 14. Either anti-C5 antibody BB5.1 mAb (20, 40, or 60 mg/kg) or vehicle was intravenously administered 60 minutes before LPS/D-GaIN administration. To avoid hypoglycemia and electrolyte imbalance, subcutaneous injections of solution containing 10% glucose water mixed with lactate ringer (25 mL/kg) were planned every 12 hours after the challenge. After 6 hours, 12 hours, 24 hours, and 72 hours, the mice were sacrificed and plasma Clq, Ba, C5a and C5b-9 Terminal Complement Complex C5b-9 were evaluated by ELISA. In addition, markers of parenchymal damage (i.e., AST, ALT, and T-Bil) and biochemical markers of microangiopathy (i.e., platelet count, LDH release, plasma ADAMTS13 activity, and Unusually-Large von Willebrand factor (UL-vWF) multimer) were assessed. Hepatic microcirculation was measured by Laser Doppler Flowmetry, 02C0 (oxygen to see), as described in www.lea.de/eng/indexe.html. Cytokines and chemokines (i.e., TNF-α, IL-1β, IL-6, IL-10, and CXCL-2) were also assessed. The following histological assessments were also conducted: polymorphonuclear leukocyte (PMN) infiltration, Liver Damage quantified by Suzuki's Score, TUNEL assay, and C4d immunostaining.

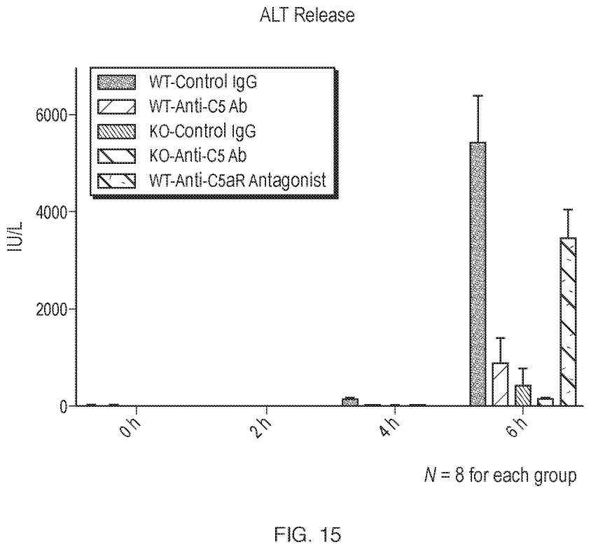

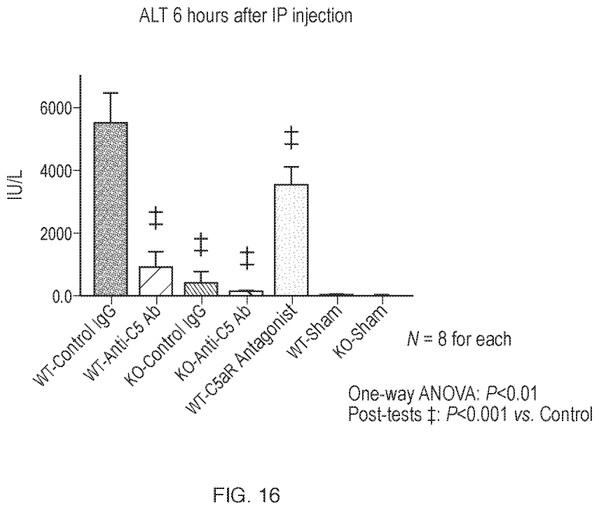

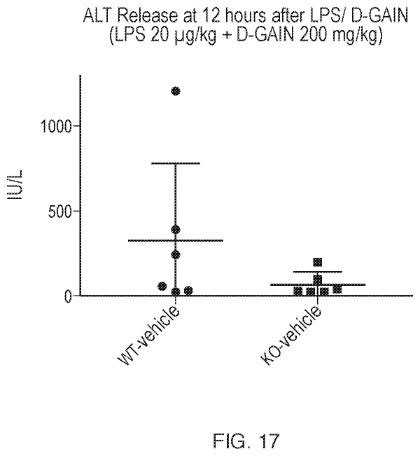

FIG. 15 depicts ALT release (IU/L) for WT-control IgG, WT-Anti-C5 Ab, KO-control IgG, KO-Anti-C5 Ab, and WT-C5aR Antagonist treated mice 0, 2, 4, and 6 hours after intraperitoneal administration of LPS (20 μg/kg) and D-GAIN (200 mg/kg). FIG. 16 depicts ALT release (IU/L) for WT-control IgG, WT-Anti-C5 Ab, KO-control IgG, KO-Anti-C5 Ab, WT-C5aR Antagonist, WT sham, and KO sham treated mice 6 hours after intraperitoneal administration of LPS (20 μg/kg) and D-GAIN (200 mg/kg). FIG. 17 depicts ALT release (IU/L) for KO- and WT-vehicle treated mice 12 hours after administration of LPS (20 μg/kg) and D-GAIN (200 mg/kg). As evidenced by FIGS. 15-17, both the anti-C5 antibody and C5-knockout significantly ameliorated liver injury at 6 hours after LPS/D-GAIN injection. In contrast, the C5aR Antagonist was not as effective as total C5 inhibition in reducing liver injury.

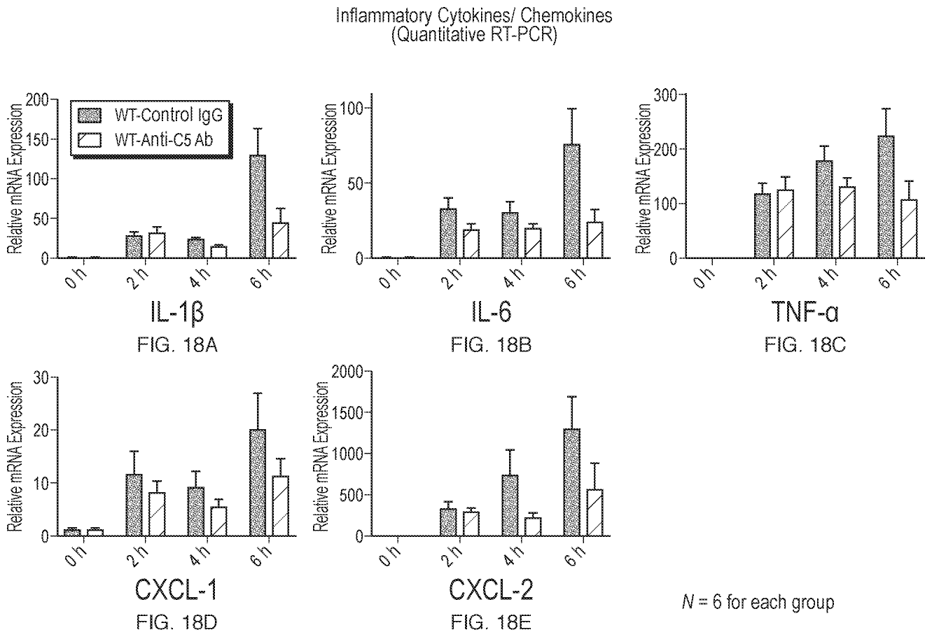

FIGS. 18A-18E depict levels of IL-10, IL-6, TNFα, CXCL-1, and CXCL-2 in WT-control and WT-Anti-C5 Ab treated mice. In brief, cytokines/chemokines started to increase as early as 2 hours after LPS/D-GAIN injection.

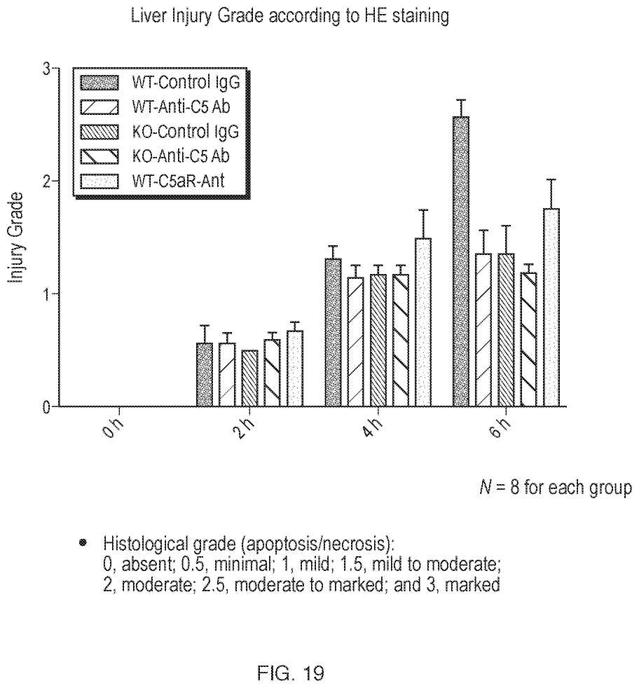

FIG. 19 is a comparison of the injury grade of WT-control IgG, WT-Anti-C5 Ab, KO-control IgG, KO-Anti-C5 Ab, and WT-C5aR-Ant treated mice as assessed via histological analysis. Histological grade (apoptosis/necrosis): 0 [absent], 0.5 [minimal], 1 [mild], 1.5 [mild to moderate], 2 [moderate], 2.5 [moderate to marked], and 3 [marked]. As shown in FIG. 19, both the anti-C5 antibody and C5-knockout significantly ameliorated liver injury at 6 hours after LPS/injection.

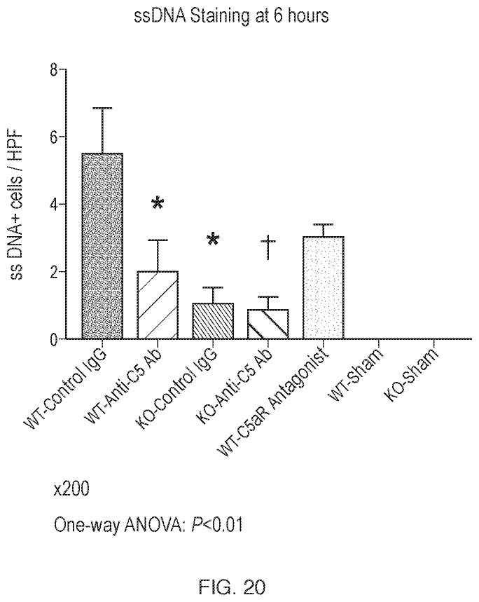

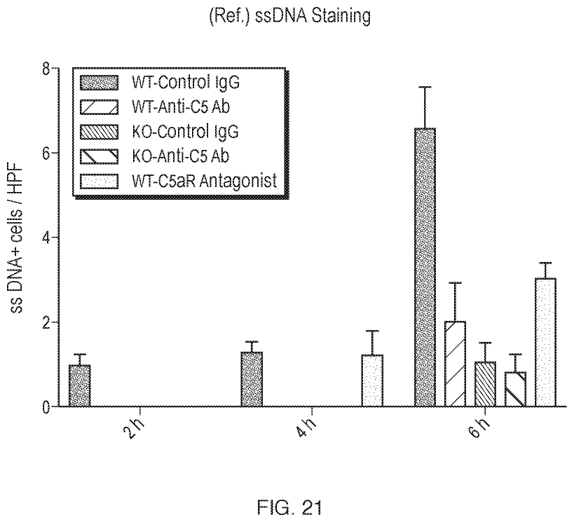

FIG. 20 depicts depicts ssDNA staining for WT-control IgG, WT-Anti-C5 Ab, KO-control IgG, KO-Anti-C5 Ab, WT-C5aR Antagonist, WT Sham, and KO Sham treated mice at 6 hours. As shown in FIG. 20, both the anti-C5 antibody and C5-knockout significantly suppressed apoptosis. FIG. 21 depicts ssDNA staining for WT-control IgG, WT-Anti-C5 Ab, KO-control IgG, KO-Anti-C5 Ab, and WT-C5aR Antagonist, at 2 hours, 4 hours, and 6 hours. As shown in FIG. 21, apoptotic change became remarkable at 6 hours in the control group. This change was suppressed by C5/C5a inhibition.

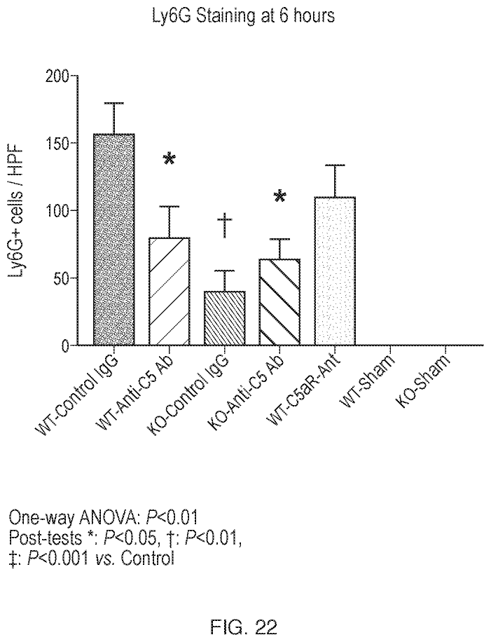

FIG. 22 depicts Ly6G staining for WT-control IgG, WT-Anti-C5 Ab, KO-control IgG, KO-Anti-C5 Ab, WT-C5aR Antagonist, WT Sham, and KO Sham treated mice at 6 hours. As evidenced by FIG. 22, both the anti-C5 antibody and C5-knockout significantly suppressed neutrophil infiltration.

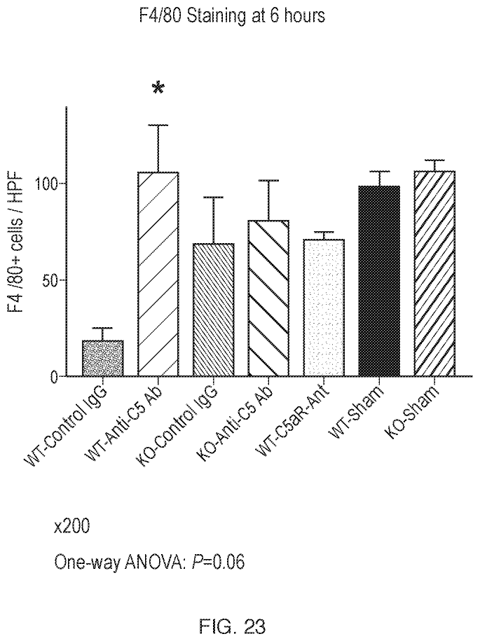

FIG. 23 depicts F4/80 staining for WT-control IgG, WT-Anti-C5 Ab, KO-control IgG, KO-Anti-C5 Ab, WT-C5aR Antagonist, WT Sham, and KO Sham treated mice at 6 hours. As evidenced by FIG. 23, both the anti-C5 antibody and C5-knockout maintained F4/80+ cells.

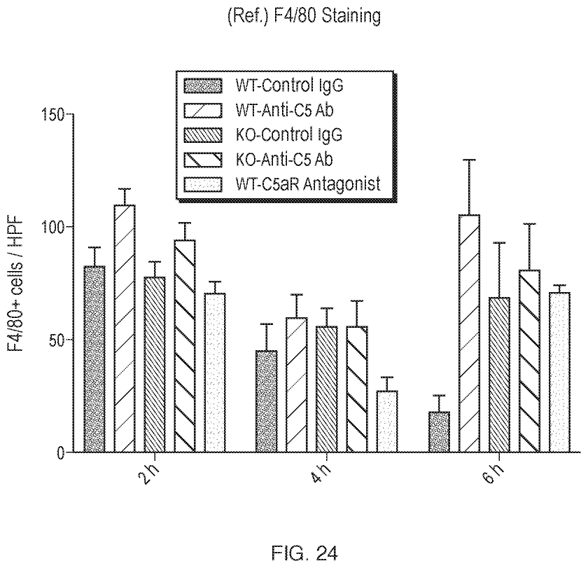

FIG. 24 depicts F4/80 staining for WT-control IgG, WT-Anti-C5 Ab, KO-control IgG, KO-Anti-C5 Ab, and WT-C5aR Antagonist at 2, 4, and 6 hours. As evidenced by FIG. 24, F4/80+ cells progressively decreased until 6 hours in the control groups.

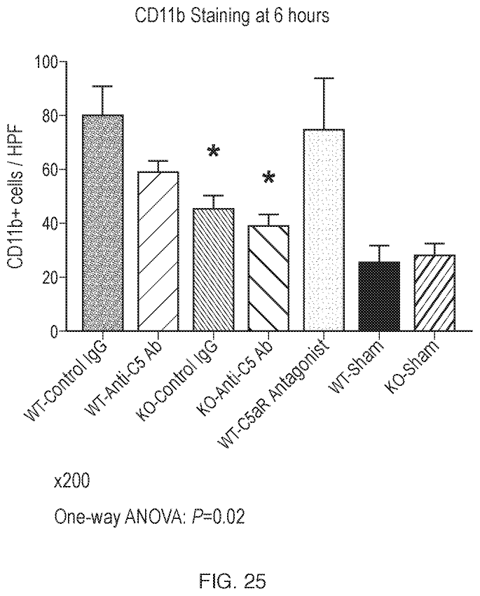

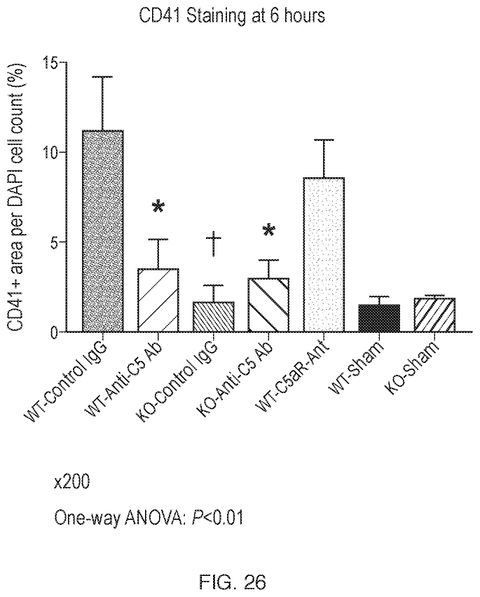

FIG. 25 depicts CD11b staining for WT-control IgG, WT-Anti-C5 Ab, KO-control IgG, KO-Anti-C5 Ab, WT-C5aR Antagonist, WT Sham, and KO Sham treated mice at 6 hours. As evidenced by FIG. 26, C5-knockout suppressed CD11b+ cells.

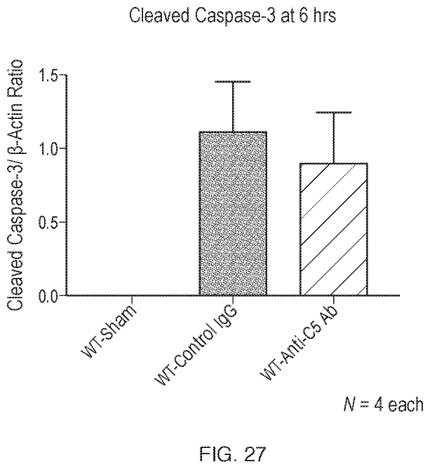

FIG. 26 depicts CD411 staining for WT-control IgG, WT-Anti-C5 Ab, KO-control IgG, KO-Anti-C5 Ab, WT-C5aR Antagonist, WT Sham, and KO Sham treated mice at 6 hours. As evidenced by FIG. 27, platelet-plug formation in sinusoids were significantly decreased by both the anti-C5 antibody and C5-knockout.

FIG. 27 depicts cleaved caspase-3/β-actin ratios for WT-sham, WT-control IgG, and WT-Anti-C5 Ab treated mice at 6 hours.

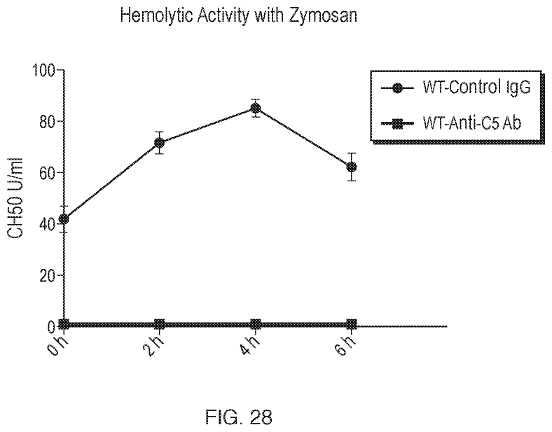

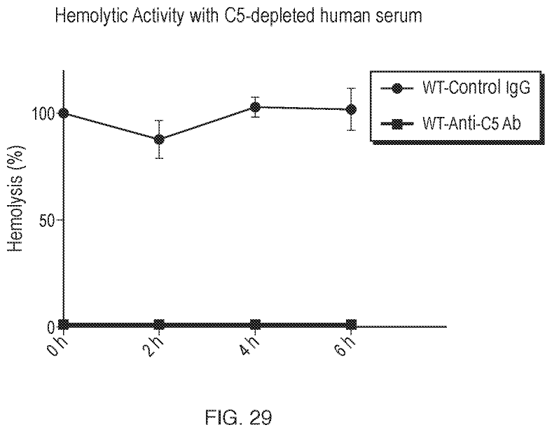

FIG. 28 depicts hemolytic activity with Zymosan for WT-control IgG and WT-Anti-C5 Ab treated mice at 2, 4, and 6 hours. FIG. 29 depicts hemolytic activity with C5-depleted human serum for WT-control IgG and WT-Anti-C5 Ab treated mice at 2, 4, and 6 hours. As evidenced by FIGS. 28-29, administration of the anti-C5 antibody completely inhibited hemolytic activity in mice.

In addition, membrane attack complex (MAC) staining was assessed at 6 hours. MAC deposition was not observed in WT-Anti-C5 Ab treated mice at 6 hours (data not shown).

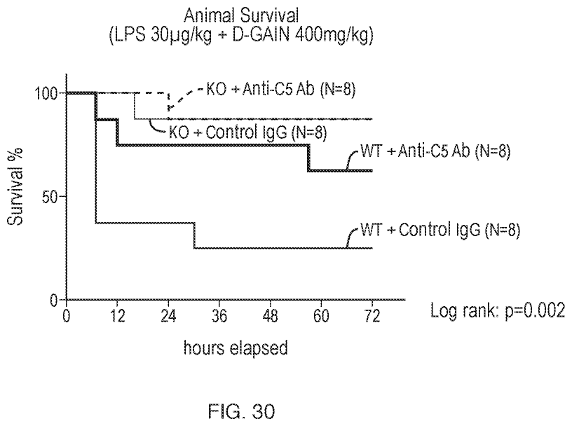

FIG. 30 depicts survival curves for KO-Anti-C5 Ab, KO-control IgG, WT-Anti-C5 Ab, and WT-control IgG treated mice over time. As evidenced by FIG. 30, both the anti-C5 antibody and C5-knockout significantly improved mouse survival.

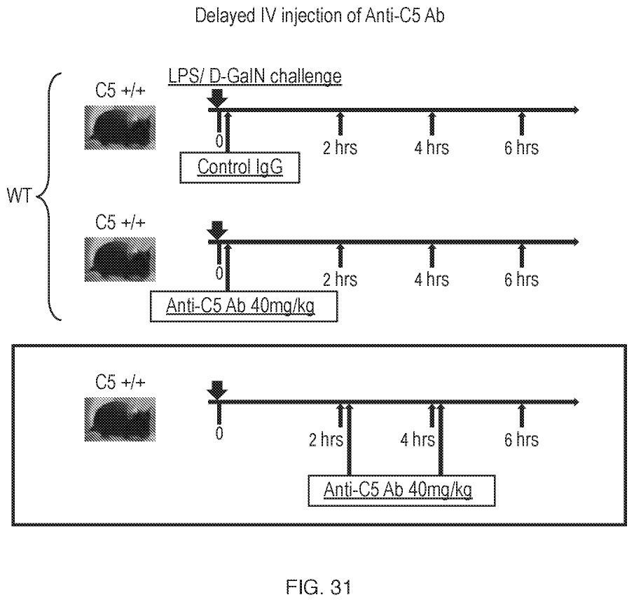

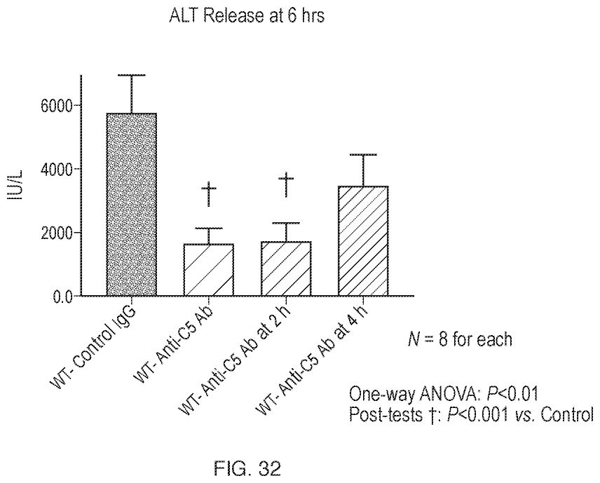

FIG. 31 is a schematic depicting the murine model of acute/fulminant liver failure, wherein intravenous injection of the Anti-C5 Ab is delayed two hours after LPS/D-GaIN injection. FIG. 32 depicts ALT release (IU/L) for WT-control IgG, WT-Anti-C5 Ab, WT-Anti-C5 Ab (administered at 2 hours), and WT-Anti-C5 Ab (administered at 4 hours) treated mice after intraperitoneal administration of LPS (20 μg/kg) and D-GAIN (200 mg/kg).

In summary, anti-C5 antibody significantly attenuated acute liver failure after LPS D-GaIN injection. Liver injury was evident 6 hours after injection and cytokines/chemokines were upregulated as early as two hours after injection.

### 3. Influence of C5 Blockade on Liver Regeneration in 70% Partial Hepatectomy in Mice

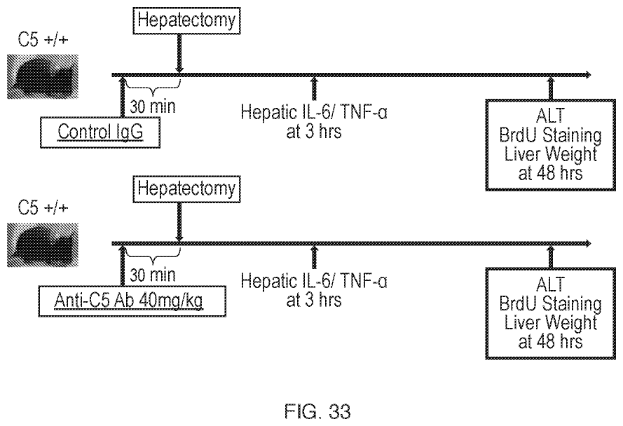

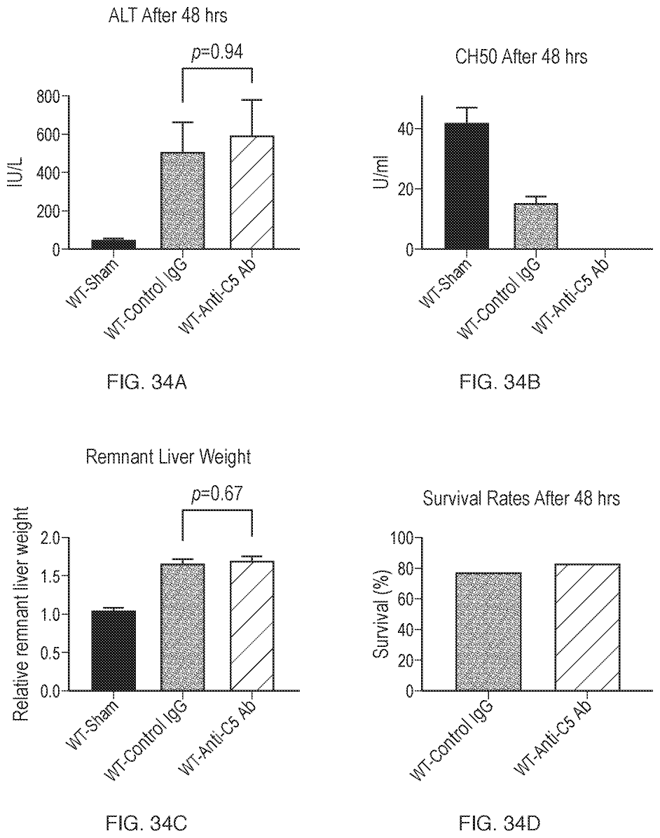

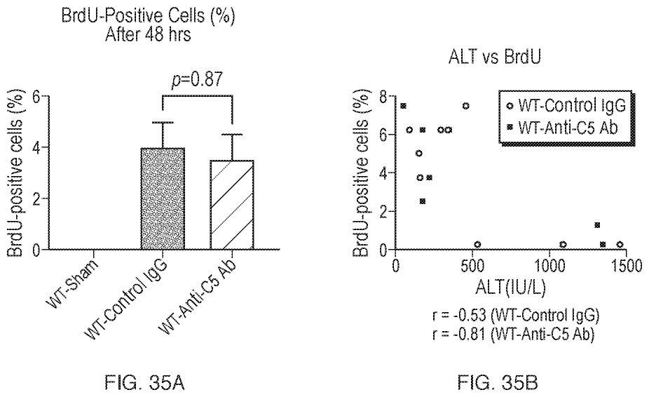

C5-knockout (KO, B10D2/oSn) and the corresponding wild-type (B10D2/nSn) mice were used. Mice were intravenously administered either control IgG or 40 mg/kg of an anti-C5 antibody (BB5.1 mAb), thirty minutes prior to receiving a 70% hepatectomy, as shown in FIG. 33. Blood/tissue samples were obtained at 2 hours, 6 hours, and 24 hours after reperfusion. Hepatic IL-6 and TNFα were measured at three hours. ALT (FIG. 34A) and CH50 (FIG. 34B) were assessed, liver weight was measured (FIG. 34C), and survival (FIG. 34D) was assessed at 48 hours. Bromodeoxyuridine (also known as BRdU, 5-bromo-2′-deoxyuridine, BrdU, BUdR, BrdUrd, and broxuridine) staining was also performed. BRdU is a synthetic nucleoside that is an analog of thymidine. BrdU is commonly used in the detection of proliferating cells in living tissues. FIG. 35A depicts the percentage of BrdU-positive cells after 48 hours for the wild-type sham, wild-type control IgG, and wild-type anti-C5 antibody groups. FIG. 35B depicts ALT (IU/L) versus % BRdU-positive cells after 48 hours.

In summary, CF-blockade by BB5.1 did not negatively affect liver regeneration. Liver regeneration was inversely proportional to liver damage in both control and BB5.1 groups.

### Example 2: Pre-Clinical Studies in Rats

The following additional pre-clinical animal studies are conducted in rats C5.

### 1. Rat Model of Orthotopic Whole Liver Transplantation

Male Lewis rats (250-300 g) are used as donors and recipients. Whole livers from donor rats are retrieved, flushed, and then stored at 4° C. in University of Wisconsin solution (1.1 W) for 24 hours, and then transplanted to recipients (Lewis-to-Lewis) with revascularization using Kamada's cuff technique without arterialization. In the recipient, either ATM602 mAb (20 mg/kg) or the vehicle (normal saline) is intravenously administrated twice (5 and 60 min before reperfusion) via penile vein. After 2 hours, 6 hours, and 24 hours of reperfusion, rats are sacrificed and blood and liver samples are collected.

Plasma Clq, Ba, C5a and C5b-9 Terminal Complement Complex C5b-9 are evaluated by ELISA. In addition, markers of parenchymal damage (i.e., AST, ALT, and T-Bil) and biochemical markers of microangiopathy (i.e., platelet count, LDH release, plasma ADAMTS13 activity, and Unusually-Large von Willebrand factor (UL-vWF) multimer) are assessed. Hepatic microcirculation is measured by Laser Doppler Flowmetry, 02C0 (oxygen to see), as described in www.lea.de/eng/indexe.html. Cytokines and chemokines (i.e., TNF-α, IL-1β, IL-6, IL-10, and CXCL-2) are also assessed. The following histological assessments are also conducted: polymorphonuclear leukocyte (PMN) infiltration, Liver Damage quantified by Suzuki's Score, TUNEL assay, and C4d immunostaining.

### 2. Rat Model of 20% Partial Liver Transplantation

In this procedure, right superior, inferior, and total paracaval liver lobes are used as a liver graft, which is approximately 20% to the total liver volume, mimicking the adult-to-adult living donor liver transplantation (LDLT) in clinical practice. Partial liver grafts are retrieved, flushed, and then stored at 4° C. in HTK solution for 6 hours, and then transplanted to Lewis rats with revascularization using Kamada's cuff technique without arterialization. In the recipient rat, either ATM602 mAb (20 mg/kg) or the vehicle (normal saline) is intravenously administrated twice (5 and 60 min before reperfusion) via penile vein. After 2 hours, 6 hours, 24 hours, and 72 hours of reperfusion, rats are sacrificed and blood and liver samples are collected.

Plasma Clq, Ba, C5a and C5b-9 Terminal Complement Complex C5b-9 are evaluated by ELISA. In addition, markers of parenchymal damage (i.e., AST, ALT, and T-Bil) and biochemical markers of microangiopathy (i.e., platelet count, LDH release, plasma ADAMTS13 activity, and Unusually-Large von Willebrand factor (UL-vWF) multimer) are assessed. Hepatic microcirculation is measured by Laser Doppler Flowmetry, 02C0 (oxygen to see), as described in www.lea.de/eng/indexe.html. Cytokines and chemokines (i.e., TNF-α, IL-1β, IL-6, IL-10, and CXCL-2) are also assessed. The following histological assessments are also conducted: polymorphonuclear leukocyte (PMN) infiltration, Liver Damage quantified by Suzuki's Score, TUNEL assay, and C4d immunostaining. Animal survival is assessed through post-operative day 10.

### 3. Rat Model of Acute/Fulminant Liver Failure:

To confirm the protective effect of C5 inhibition in progression of acute liver failure, a rat model is employed. Male Lewis rats (200-250 g) are subjected to thioacetamide (TAA) challenge (400 mg/kg, i.p. twice). Either ATM602 mAb (20, 40, or 60 mg/kg) or vehicle is intravenously administered 60 minutes before TAA administration. To avoid hypoglycemia and electrolyte imbalance, subcutaneous injections of solution containing 10% glucose water mixed with lactate ringer (25 0 mL/kg) is given every 12 hours after the TA challenge. After 12 hours, 24 hours, and 72 hours, the rats are sacrificed and plasma Clq, Ba, C5a and C5b-9 Terminal Complement Complex C5b-9 are evaluated by ELISA. In addition, markers of parenchymal damage (i.e., AST, ALT, and T-Bil) and biochemical markers of microangiopathy (i.e., platelet count, LDH release, plasma ADAMTS13 activity, and Unusually-Large von Willebrand factor (UL-vWF) multimer) are assessed. Hepatic microcirculation is measured by Laser Doppler Flowmetry, 02C0 (oxygen to see), as described in www.lea.de/eng/indexe.html. Cytokines and chemokines (i.e., TNF-α, IL-1β, IL-6, IL-10, and CXCL-2) are also assessed. The following histological assessments are also conducted: polymorphonuclear leukocyte (PMN) infiltration, Liver Damage quantified by Suzuki's Score, TUNEL assay, and C4d immunostaining.

SEQUENCE SUMMARY

SEQ ID NO: 1
GYIFSNYWIQ
SEQ ID NO: 2
EILPGSGSTEYTENFKD
SEQ ID NO: 3
YFFGSSPNWYFDV
SEQ ID NO: 4
GASENIYGALN
SEQ ID NO: 5
GATNLAD
SEQ ID NO: 6
QNVLNTPLT
SEQ ID NO: 7
QVQLVQSGAEVKKPGASVKVSCKASGYIFSNYWIQWVRQAPGQGLEWMGE
ILPGSGSTEYTENFKDRVTMTRDTSTSTVYMELSSLRSEDTAVYYCARYF
FGSSPNWYFDVWGQGTLVTVSS
SEQ ID NO: 8
DIQMTQSPSSLSASVGDRVTITCGASENIYGALNWYQQKPGKAPKWYGAT
NLADGVPSRFSGSGSGTDFTLTISSLQPEDFATYYCQNVLNTPLTFGQGT
KVEIK
SEQ ID NO: 9
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSQEDP
EVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGN
VFSCSVMHEALHNHYTQKSLSLSLGK
SEQ ID NO: 10
QVQLVQSGAEVKKPGASVKVSCKASGYIFSNYWIQWVRQAPGQGLEWMGE
ILPGSGSTEYTENFKDRVTMTRDTSTSTVYMELSSLRSEDTAVYYCARYF
FGSSPNWYFDVWGQGTLVTVSSASTKGPSVFPLAPCSRSTSESTAALGCL
VKDYFPEPVTVSWNSGALTSGVHTFPAVLQSSGLYSLSSVVTVPSSNFGT
QTYTCNVDHKPSNTKVDKTVERKCCVECPPCPAPPVAGPSVFLFPPKPKD
TLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLGK
SEQ ID NO: 11
DIQMTQSPSSLSASVGDRVTITCGASENIYGALNWYQQKPGKAPKWYGAT
NLADGVPSRFSGSGSGTDFTLTISSLQPEDFATYYCQNVLNTPLTFGQGT
KVEIKRTVAAPSVFIFPPSDEQLKSGTASVVCLLNNFYPREAKVQWKVDN
ALQSGNSQESVTEQDSKDSTYSLSSTLTLSKADYEKHKVYACEVTHQGLS
SPVTKSFNRGEC
SEQ ID NO: 12
QVQLVQSGAEVKKPGASVKVSCKASGHIFSNYWIQWVRQAPGQGLEWMGE
ILPGSGHTEYTENFKDRVTMTRDTSTSTVYMELSSLRSEDTAVYYCARYF
FGSSPNWYFDVWGQGTLVTVSS
SEQ ID NO: 13
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSQEDP
EVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGN
VFSCSVLHEALHSHYTQKSLSLSLGK
SEQ ID NO: 14
QVQLVQSGAEVKKPGASVKVSCKASGHIFSNYWIQWVRQAPGQGLEWMGE
ILPGSGHTEYTENFKDRVTMTRDTSTSTVYMELSSLRSEDTAVYYCARYF
FGSSPNWYFDVWGQGTLVTVSS ASTKGPSVFPLAPCSRSTSESTAALGC
LVKDYFPEPVTVSWNSGALTSGVHTFPAVLQSSGLYSLSSVVTVPSSNFG
TQTYTCNVDHKPSNTKVDKTVERKCCVECPPCPAPPVAGPSVFLFPPKPK
DTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQV
YTLPPSQEEMTKNQVSLTCLVKGFYPSDIAVEWESNGQPENNYKTTPPVL
DSDGSFFLYSRLTVDKSRWQEGNVFSCSVLHEALHSHYTQKSLSLSLGK
SEQ ID NO: 15
ASTKGPSVFPLAPCSRSTSESTAALGCLVKDYFPEPVTVSWNSGALTSGV
HTFPAVLQSSGLYSLSSVVTVTSSNFGTQTYTCNVDHKPSNTKVDKTVER
KCCVECPPCPAPPVAGPSVFLFPPKPKDTLYITREPEVTCVVVDVSHEDP
EVQFNWYVDGMEVHNAKTKPREEQFNSTFRVVSVLTVVHQDWLNGKEYKC
KVSNKGLPAPIEKTISKTKGQPREPQVYTLPPSREEMTKNQVSLTCLVKG
FYPSDIAVEWESNGQPENNYKTTPPMLDSDGSFFLYSKLTVDKSRWQQGN
VFSCSVMHEALHNHYTQKSLSLSPGK
SEQ ID NO: 16
QVQLVQSGAEVKKPGASVKVSCKASGYIFSNYWIQWVRQAPGQGLEWMGE
ILPGSGSTEYTENFKDRVTMTRDTSTSTVYMELSSLRSEDTAVYYCARYF
FGSSPNWYFDVWGQGTLVTVSSASTKGPSVFPLAPCSRSTSESTAALGCL
VKDYFPEPVTVSWNSGALTSGVHTFPAVLQSSGLYSLSSVVTVTSSNFGT
QTYTCNVDHKPSNTKVDKTVERKCCVECPPCPAPPVAGPSVFLFPPKPKD
TLYITREPEVTCVVVDVSHEDPEVQFNWYVDGMEVHNAKTKPREEQFNST
FRVVSVLTVVHQDWLNGKEYKCKVSNKGLPAPIEKTISKTKGQPREPQVY
TLPPSREEMTKNQVSLTCLVKGFYPSDIAVEWESNGQPENNYKTTPPMLD
SDGSFFLYSKLTVDKSRWQQGNVFSCSVMHEALHNHYTQKSLSLSPGK
SEQ ID NO: 17
GASENIYHALN
SEQ ID NO: 18
EILPGSGHTEYTENFKD
SEQ ID NO: 19
GHIFSNYWIQ
SEQ ID NO: 20
QVQLVQSGAEVKKPGASVKVSCKASGHIFSNYWIQWVRQAPGQGLEWMGE
ILPGSGHTEYTENFKDRVTMTRDTSTSTVYMELSSLRSEDTAVYYCARYF
FGSSPNWYFDVWGQGTLVTVSSASTKGPSVFPLAPCSRSTSESTAALGCL
VKDYFPEPVTVSWNSGALTSGVHTFPAVLQSSGLYSLSSVVTVPSSNFGT
QTYTCNVDHKPSNTKVDKTVERKCCVECPPCPAPPVAGPSVFLFPPKPKD
TLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLGK
SEQ ID NO: 21
SYAIS
SEQ ID NO: 22
GIGPFFGTANYAQKFQG
SEQ ID NO: 23
DTPYFDY
SEQ ID NO: 24
SGDSIPNYYVY
SEQ ID NO: 25
DDSNRPS
SEQ ID NO: 26
QSFDSSLNAEV
SEQ ID NO: 27
QVQLVQSGAEVKKPGSSVKVSCKASGGTFSSYAISVWRQAPGQGLEWMGG
IGPFFGTANYAQKFQGRVTITADESTSTAYMELSSLRSEDTAVYYCARDT
PYFDYWGQGTLVTVSS
SEQ ID NO: 28
DIELTQPPSVSVAPGQTARISCSGDS1PNYYVYWYQQKPGQAPVLVIYDD
SNRPSGIPERFSGSNSGNTATLTISGTQAEDEADYYCQSFDSSLNAEVFG
GGTK LTVL
SEQ ID NO: 29
NYIS
SEQ ID NO: 30
IIDPDDSYTEYSPSFQG
SEQ ID NO: 31
YEYGGFDI
SEQ ID NO: 32
SGDNIGNSYVH
SEQ ID NO: 33
KDNDRPS
SEQ ID NO: 34
GTYDIESYV
SEQ ID NO: 35
EVQLVQSGAEVKKPGESLKISCKGSGYSFTNYISWVRQMPGKGLEWMGII
DPDDSYTEYSPSFQGQVTISADKSISTAYLQWSSLKASDTAMYYCARYEY
GGFDIWGQGTLVTVSS
SEQ ID NO: 36
SYELTQPPSVSVAPGQTARISCSGDNIGNSYVHWYQQKPGQAPVLVIYKD
NDRPSGIPERFSGSNSGNTATLTISGTQAEDEADYYCGTYDIESYVFGGG
TKLTV L
SEQ ID NO: 37
SSYYVA
SEQ ID NO: 38
AIYTGSGATYKASWAKG
SEQ ID NO: 39
DGGYDYPTHAMHY
SEQ ID NO: 40
QASQNIGSSLA
SEQ ID NO: 41
GASKTHS
SEQ ID NO: 42
QSTKVGSSYGNH
SEQ ID NO: 43
QVQLVESGGGLVQPGGSLRLSCAASGFTSHSSYYVAWVRQAPGKGLEWVG
AIYTGSGATYKASWAKGRFTISKDTSKNQVVLTMTNMDPVDTATYYCASD
GGYDYPTHAMHYWGQGTLVTVSS
SEQ ID NO: 44
DVVMTQSPSSLSASVGDRVTITCQASQNIGSSLAWYQQKPGQAPRLLIYG
ASKTHSGVPSRFSGSGSGTDFTLTISSLQPEDVATYYCQSTKVGSSYGNH
FGGGTKVEIK
SEQ ID NO: 45
QVQLVESGGGLVQPGRSLRLSCAASGFTVHSSYYMAWVRQAPGKGLEWVG
AIFTGSGAEYKAEWAKGRVTISKDTSKNQVVLTMTNMDPVDTATYYCASD
AGYDYPTHAMHYWGQGTLVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKKVEPKSCDKTHTCPPCPAPELRRGPKVFLFP
PKPKDTLMISRTPEVTCVVVDVSHEDPEVKFNWYVDGVEVHNAKTKPREE
QYNSTYRVVSVLTVLHQDWLNGKEYKCKVSNKGLPSSIEKTISKAKGQPR
EPQVYTLPPSREEMTKNQVSLTCLVKGFYPSDIAVEWESNGQPENNYKTT
PPVLDSDGSFFLYSKLTVDKSRWQQGNVFSCSVLHEALHAHYTRKELSLS
P
SEQ ID NO: 46
DIQMTQSPSSLSASVGDRVTITCRASQGISSSLAWYQQKPGKAPKLLIYG
ASETESGVPSRFSGSGSGTDFTLTISSLQPEDFATYYCQNTKVGSSYGNT
FGGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEVT
HQGLSSPVTKSFNRGEC
SEQ ID NO: 47
QVQLQESGPGLVKPSETLSLTCTVSGDSVSSSYWTWIRQPPGKGLEWIGY
IYYSGSSNYNPSLKSRATISVDTSKNQFSLKLSSVTAADTAVYYCAREGN
VDTTMIFDYWGQGTLVTVSS
SEQ ID NO: 48
AIQMTQSPSSLSASVGDRVTITCRASQGIRNDLGWYQQKPGKAPKLLIYA
ASSLQSGVPSRFAGRGSGTDFTLTISSLQPEDFATYYCLQDFNYPWTFGQ
GTKVEIK
SEQ ID NO: 49
QVQLQESGPGLVKPSETLSLTCTVSGDSVSSSYWTWIRQPPGKGLEWIGY
IYYSGSSNYNPSLKSRATISVDTSKNQFSLKLSSVTAADTAVYYCAREGN
VDTTMIFDYWGQGTLVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK
SEQ ID NO: 50
AIQMTQSPSSLSASVGDRVTITCRASQGIRNDLGWYQQKPGKAPKLLIYA
ASSLQSGVPSRFAGRGSGTDFTLTISSLQPEDFATYYCLQDFNYPWTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

1. A method of treating hepatic ischemia reperfusion injury (IRI) in a patient who has experienced hepatic trauma, the method comprising administering to the patient an effective amount of an anti-C5 agent.

2. The method of claim 1, wherein the treatment results in:
(a) a decrease in one or more pro-inflammatory cytokines and/or one or more chemokines compared to a pre-treatment baseline;
(b) a decrease in one or more of IL-1, IL-6, TNFα, CXCL-1 and CXCL-2 compared to a pre-treatment baseline;
(c) a decrease in one or more parenchymal damage markers selected from the group consisting of AST, ALT and T-bil compared to a pre-treatment baseline;
(d) decreased neutrophil infiltration and/or platelet aggregation compared to a pre-treatment baseline;
(e) an at least one score improvement according to the Suzuki Scoring System; and/or
(f) in decreased hepatocyte apoptosis compared to a pre-treatment baseline, as assessed before and/or after treatment by single-stranded-DNA staining and/or western blot for cleaved caspase-3.

3-8. (canceled)

9. A method of decreasing levels of one or more pro-inflammatory cytokines and/or one or more chemokines in a patient who has experienced hepatic trauma, the method comprising administering to the patient an effective amount of an anti-C5 agent, thereby decreasing levels of the one or more pro-inflammatory cytokines and/or one or more chemokines in the patient compared to pre-treatment baseline levels.

10. The method of claim 9, wherein (a) the one or more pro-inflammatory cytokine is selected from the group consisting of IL-1, IL-6, and TNFα and/or (b) the one or more chemokines is CXCL-1 and/or CXCL-2.

11. (canceled)

12. A method of decreasing neutrophil infiltration in a patient who has experienced hepatic trauma, the method comprising administering to the patient an effective amount of an anti-C5 agent, thereby decreasing neutrophil levels compared to pre-treatment baseline neutrophil levels.

13. A method of treating a patient who has been determined to have acute liver failure, the method comprising administering to the patient an effective amount of an anti-C5 agent.

14. The method of claim 13, wherein the treatment results in:
(a) a shift towards normal levels of serum albumin;
(b) a Prothrombin Time (PT) between 9.5 to 13.5 seconds and/or an International Normalized Ratio (INR) between 0.8 to 1.1;
(c) at least one therapeutic effect selected from the group consisting of a reduction or cessation in hepatic encephalopathy, impaired protein synthesis, jaundice, pain in the upper right abdomen, abdominal swelling, nausea, vomiting, malaise, disorientation, confusion, and/or sleepiness; and/or
(d) a change from baseline, as assessed via The King's College criteria system, the Model for End-Stage Liver Disease (MELD) scoring system, the Acute Physiology and Chronic Health Evaluation (APACHE) II scoring system, and/or the Clichy criteria.

15-17. (canceled)

18. A method of increasing serum albumin in a patient who has been determined to have acute liver failure, the method comprising administering to the patient an effective amount of an anti-C5 agent, thereby increasing serum albumin in the patient compared to a pre-administration baseline serum albumin level.

19. The method of claim 18, wherein the patient's serum albumin is:
(a) below 3.4 grams per deciliter prior to administration of the anti-C5 agent; and/or
(b) between 3.4 grams to 5.4 grams per deciliter after administration of the anti-C5 agent.

20. (canceled)

21. A method of decreasing Prothrombin Time (PT) in a patient who has been determined to have acute liver failure, the method comprising administering to the patient an effective amount of an anti-C5 agent, thereby decreasing PT time in the patient compared to the patient's pre-administration PT.

22. The method of claim 21, wherein the patient's PT is:
(a) >13.5 seconds prior to administration of the anti-C5 agent; and/or
(b) between is 9.5 to 13.5 seconds after administration of the anti-C5 agent.

23. (canceled)

24. A method of decreasing International Normalized Ratio (INR) in a patient who has been determined to have acute liver failure, the method comprising administering to the patient an effective amount of an anti-C5 agent, thereby decreasing INR in the patient compared to the patient's pre-administration INR.

25. The method of claim 24, wherein the patient's INR is:
(a) >1.5 prior to administration of the anti-C5 agent; and/or
(b) between 0.8 to 1.1 after administration of the anti-C5 agent.

26. (canceled)

27. The method of claim 24, wherein the decrease is assessed 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 48, or 72 hours post treatment.

28. The method of claim 1, wherein the anti-C5 agent is an anti-C5 antibody, or antigen binding fragment thereof, and wherein the antibody, or antigen binding fragment thereof:
(a) comprises CDR1, CDR2, and CDR3 heavy chain sequences as set forth in SEQ ID NOs:1, 2, and 3, respectively, and CDR1, CDR2, and CDR3 light chain sequences as set forth in SEQ ID NOs:4, 5, and 6, respectively;
(b) comprises a heavy chain variable region comprising SEQ ID NO:7 and a light chain variable region comprising SEQ ID NO:8;
(c) comprises a heavy chain comprising SEQ ID NO:10 and a light chain comprising SEQ ID NO:11; and/or
(d) is eculizumab.

29. The method of claim 1, wherein the anti-C5 agent is an anti-C5 antibody, or antigen binding fragment thereof, and wherein the antibody, or antigen binding fragment thereof:
(a) comprises CDR1, CDR2, and CDR3 heavy chain sequences as set forth in SEQ ID NOs:19, 18, and 3, respectively, and CDR1, CDR2, and CDR3 light chain sequences as set forth in SEQ ID NOs:4, 5, and 6, respectively;
(b) comprises a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434 of a native human IgG Fc constant region, each in EU numbering;
(c) comprises a heavy chain variable region comprising SEQ ID NO:12 and a light chain variable region comprising SEQ ID NO:8;
(d) comprises a heavy chain constant region depicted in SEQ ID NO:13;
(e) comprises a heavy chain polypeptide comprising the amino acid sequence depicted in SEQ ID NO:14 and a light chain polypeptide comprising the amino acid sequence depicted in SEQ ID NO:11; and/or
(f) is ravulizumab.

30-38. (canceled)

39. The method of claim 1, wherein the anti-C5 agent is an anti-C5 antibody, or antigen binding fragment thereof, and wherein the antibody, or antigen-binding fragment thereof, comprises:
(a) heavy chain CDR1, CDR2 and CDR3 domains having the sequences set forth in SEQ ID NOs: 21, 22, and 23, respectively, and light chain CDR1, CDR2 and CDR3 domains having the sequences set forth in SEQ ID NOs: 24, 25, and 26, respectively;
(b) a heavy chain variable region comprising the sequence set forth in SEQ ID NO:27 and a light chain variable region having the sequence set forth in SEQ ID NO:28;
(c) heavy chain CDR1, CDR2 and CDR3 domains having the sequences set forth in SEQ ID NOs: 29, 30, and 31, respectively, and light chain CDR1, CDR2 and CDR3 domains having the sequences set forth in SEQ ID NOs: 32, 33, and 34, respectively;
(d) a heavy chain variable region comprising the sequence set forth in SEQ ID NO:35 and a light chain variable region having the sequence set forth in SEQ ID NO:36;
(e) heavy chain CDR1, CDR2 and CDR3 domains having the sequences set forth in SEQ ID NOs: 37, 38, and 39, respectively, and light chain CDR1, CDR2 and CDR3 domains having the sequences set forth in SEQ ID NOs: 40, 41, and 42, respectively;
(f) a heavy chain variable region comprising the sequence set forth in SEQ ID NO:43 and a light chain variable region having the sequence set forth in SEQ ID NO:44;
(g) a heavy chain comprising the sequence set forth in SEQ ID NO: 45 and a light chain comprising the sequence set forth in SEQ ID NO: 46;
(h) a heavy chain variable region sequence set forth in SEQ ID NO: 47 and a light chain variable region comprising the sequence set forth in SEQ ID NO: 48; or
(i) a heavy chain sequence set forth in SEQ ID NO: 49 and a light chain sequence set forth in SEQ ID NO: 50.

40-47. (canceled)

48. The method of claim 1, wherein the anti-C5 agent is administered intravenously.

49. A kit for treating a patient who has been determined to have acute liver failure or for treating hepatic ischemia reperfusion injury (IRI) in a patient, the kit comprising:
(a) a dose of an anti-C5 agent; and
(b) instructions for using the anti-C5 agent, in the method of claim 1.

50-51. (canceled)


SEQUENCE LISTING (50 sequences)

SEQ ID NO: 1 - protein, 10 aa, Artificial Sequence
  source: /note=“Description of Artificial Sequence Synthetic peptide”
GYIFSNYWIQ

SEQ ID NO: 2 - protein, 17 aa, Artificial Sequence
  source: /note=“Description of Artificial Sequence Synthetic peptide”
EILPGSGSTEYTENFKD

SEQ ID NO: 3 - protein, 13 aa, Artificial Sequence
  source: /note=“Description of Artificial Sequence Synthetic peptide”
YFFGSSPNWYFDV

SEQ ID NO: 4 - protein, 11 aa, Artificial Sequence
  source: /note=“Description of Artificial Sequence Synthetic peptide”
GASENIYGALN

SEQ ID NO: 5 - protein, 7 aa, Artificial Sequence
  source: /note=“Description of Artificial Sequence Synthetic peptide”
GATNLAD

SEQ ID NO: 6 - protein, 9 aa, Artificial Sequence
  source: /note=“Description of Artificial Sequence Synthetic peptide”
QNVLNTPLT

SEQ ID NO: 7 - protein, 122 aa, Artificial Sequence [sequence omitted]

SEQ ID NO: 8 - protein, 107 aa, Artificial Sequence [sequence omitted]

SEQ ID NO: 9 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 448 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 214 aa, Artificial Sequence [sequence omitted]

SEQ ID NO: 12 - protein, 122 aa, Artificial Sequence [sequence omitted]

SEQ ID NO: 13 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 14 - protein, 448 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 15 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 16 - protein, 448 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 17 - protein, 11 aa, Artificial Sequence
  source: /note=“Description of Artificial Sequence Synthetic peptide”
GASENIYHALN

SEQ ID NO: 18 - protein, 17 aa, Artificial Sequence
  source: /note=“Description of Artificial Sequence Synthetic peptide”
EILPGSGHTEYTENFKD

SEQ ID NO: 19 - protein, 10 aa, Artificial Sequence
  source: /note=“Description of Artificial Sequence Synthetic peptide”
GHIFSNYWIQ

SEQ ID NO: 20 - protein, 448 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 5 aa, Artificial Sequence
  source: /note=“Description of Artificial Sequence Synthetic peptide”
SYAIS

SEQ ID NO: 22 - protein, 17 aa, Artificial Sequence
  source: /note=“Description of Artificial Sequence Synthetic peptide”
GIGPFFGTANYAQKFQG

SEQ ID NO: 23 - protein, 7 aa, Artificial Sequence
  source: /note=“Description of Artificial Sequence Synthetic peptide”
DTPYFDY

SEQ ID NO: 24 - protein, 11 aa, Artificial Sequence
  source: /note=“Description of Artificial Sequence Synthetic peptide”
SGDSIPNYYVY

SEQ ID NO: 25 - protein, 7 aa, Artificial Sequence
  source: /note=“Description of Artificial Sequence Synthetic peptide”
DDSNRPS

SEQ ID NO: 26 - protein, 11 aa, Artificial Sequence
  source: /note=“Description of Artificial Sequence Synthetic peptide”
QSFDSSLNAEV

SEQ ID NO: 27 - protein, 116 aa, Artificial Sequence
  source: /note=“Description of Artificial Sequence Synthetic polypeptide”
QVQLVQSGAEVKKPGSSVKVSCKASGGTFSSYAISVWRQAPGQGLEWMGGIGPFFGTANYAQKFQGRVTITADESTSTAYMELSSLRSEDTAVYYCARDTPYFDYWGQGTLVTVSS

SEQ ID NO: 28 - protein, 108 aa, Artificial Sequence
  source: /note=“Description of Artificial Sequence Synthetic polypeptide”
DIELTQPPSVSVAPGQTARISCSGDSIPNYYVYWYQQKPGQAPVLVIYDDSNRPSGIPERFSGSNSGNTATLTISGTQAEDEADYYCQSFDSSLNAEVFGGGTKLTVL

SEQ ID NO: 29 - protein, 4 aa, Artificial Sequence
  source: /note=“Description of Artificial Sequence Synthetic peptide”
NYIS

SEQ ID NO: 30 - protein, 17 aa, Artificial Sequence
  source: /note=“Description of Artificial Sequence Synthetic peptide”
IIDPDDSYTEYSPSFQG

SEQ ID NO: 31 - protein, 8 aa, Artificial Sequence
  source: /note=“Description of Artificial Sequence Synthetic peptide”
YEYGGFDI

SEQ ID NO: 32 - protein, 11 aa, Artificial Sequence
  source: /note=“Description of Artificial Sequence Synthetic peptide”
SGDNIGNSYVH

SEQ ID NO: 33 - protein, 7 aa, Artificial Sequence
  source: /note=“Description of Artificial Sequence Synthetic peptide”
KDNDRPS

SEQ ID NO: 34 - protein, 9 aa, Artificial Sequence
  source: /note=“Description of Artificial Sequence Synthetic peptide”
GTYDIESYV

SEQ ID NO: 35 - protein, 116 aa, Artificial Sequence
  source: /note=“Description of Artificial Sequence Synthetic polypeptide”
EVQLVQSGAEVKKPGESLKISCKGSGYSFTNYISWVRQMPGKGLEWMGIIDPDDSYTEYSPSFQGQVTISADKSISTAYLQWSSLKASDTAMYYCARYEYGGFDIWGQGTLVTVSS

SEQ ID NO: 36 - protein, 106 aa, Artificial Sequence
  source: /note=“Description of Artificial Sequence Synthetic polypeptide”
SYELTQPPSVSVAPGQTARISCSGDNIGNSYVHWYQQKPGQAPVLVIYKDNDRPSGIPERFSGSNSGNTATLTISGTQAEDEADYYCGTYDIESYVFGGGTKLTVL

SEQ ID NO: 37 - protein, 6 aa, Artificial Sequence
  source: /note=“Description of Artificial Sequence Synthetic peptide”
SSYYVA

SEQ ID NO: 38 - protein, 17 aa, Artificial Sequence
  source: /note=“Description of Artificial Sequence Synthetic peptide”
AIYTGSGATYKASWAKG

SEQ ID NO: 39 - protein, 13 aa, Artificial Sequence
  source: /note=“Description of Artificial Sequence Synthetic peptide”
DGGYDYPTHAMHY

SEQ ID NO: 40 - protein, 11 aa, Artificial Sequence
  source: /note=“Description of Artificial Sequence Synthetic peptide”
QASQNIGSSLA

SEQ ID NO: 41 - protein, 7 aa, Artificial Sequence
  source: /note=“Description of Artificial Sequence Synthetic peptide”
GASKTHS

SEQ ID NO: 42 - protein, 12 aa, Artificial Sequence
  source: /note=“Description of Artificial Sequence Synthetic peptide”
QSTKVGSSYGNH

SEQ ID NO: 43 - protein, 123 aa, Artificial Sequence
  source: /note=“Description of Artificial Sequence Synthetic polypeptide”
QVQLVESGGGLVQPGGSLRLSCAASGFTSHSSYYVAWVRQAPGKGLEWVGAIYTGSGATYKASWAKGRFTISKDTSKNQVVLTMTNMDPVDTATYYCASDGGYDYPTHAMHYWGQGTLVTVSS

SEQ ID NO: 44 - protein, 110 aa, Artificial Sequence
  source: /note=“Description of Artificial Sequence Synthetic polypeptide”
DVVMTQSPSSLSASVGDRVTITCQASQNIGSSLAWYQQKPGQAPRLLIYGASKTHSGVPSRFSGSGSGTDFTLTISSLQPEDVATYYCQSTKVGSSYGNHFGGGTKVEIK

SEQ ID NO: 45 - protein, 451 aa, Artificial Sequence
  source: /note=“Description of Artificial Sequence Synthetic polypeptide”
QVQLVESGGGLVQPGRSLRLSCAASGFTVHSSYYMAWVRQAPGKGLEWVGAIFTGSGAEYKAEWAKGRVTISKDTSKNQVVLTMTNMDPVDTATYYCASDAGYDYPTHAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 46 - protein, 217 aa, Artificial Sequence
  source: /note=“Description of Artificial Sequence Synthetic polypeptide”
DIQMTQSPSSLSASVGDRVTITCRASQGISSSLAWYQQKPGKAPKLLIYGASETESGVPSRFSGSGSGTDFTLTISSLQPEDFATYYCQNTKVGSSYGNTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 120 aa, Artificial Sequence
  source: /note=“Description of Artificial Sequence Synthetic polypeptide”
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIFDYWGQGTLVTVSS

SEQ ID NO: 48 - protein, 107 aa, Artificial Sequence
  source: /note=“Description of Artificial Sequence Synthetic polypeptide”
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWTFGQGTKVEIK

SEQ ID NO: 49 - protein, 447 aa, Artificial Sequence
  source: /note=“Description of Artificial Sequence Synthetic polypeptide”
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - protein, 214 aa, Artificial Sequence
  source: /note=“Description of Artificial Sequence Synthetic polypeptide”
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC